(19) United States
(12) Patent Application Publication
(10) Pub. No.: US 2026/0328779 A1
(43) Pub. Date: Oct. 22, 2026

(54) METHODS AND COMPOSITIONS FOR THE DIAGNOSIS AND TREATMENT OF IGA NEPHROPATHY

(71) Applicant: Beth Israel Deaconess Medical Center, Inc., Boston, MA (US)

(72) Inventors: Richard D. Cummings, Brookline, MA (US); Elliot Chaikof, Newton, MA (US); Yasuyuki Matsumoto, Revere, MA (US); Sylvain Lehoux, Lexington, MA (US)

(73) Assignee: Beth Israel Deaconess Medical Center, Inc., Boston, MA (US)

(21) Appl. No.: 19/109,845

(22) PCT Filed: Sep. 8, 2023
(86) PCT No.: PCT/US2023/032240

§ 371 Date: Mar. 7, 2025

Related U.S. Application Data
(60) Provisional application No. 63/405,048, filed on Sep. 9, 2022.

(51) Int. Cl. G01N 33/68; A61K 31/7016; G01N 33/543

(52) U.S. Cl. CPC G01N 33/6893; A61K 31/7016; G01N 33/54313; G01N 2800/347

(57) ABSTRACT

The present disclosure provides methods and compositions for the diagnosis and treatment of IgA nephropathy. Detection and quantitation of serum IgM levels is performed using an IgA molecule including a GalNAc-alpha 1-Ser/Thr antigen. ELISAs and flow cytometry based methods are claimed. In the examples, Asialo-BSM microbeads are used.

### CROSS-REFERENCE TO RELATED APPLICATIONS

This application claims the benefit under 35 U.S.C. § 119(e) of U.S. Provisional Application No. 63/405,048, filed Sep. 9, 2022, entitled “METHODS AND COMPOSITIONS FOR THE DIAGNOSIS AND TREATMENT OF IGA NEPHROPATHY,” the entire contents of which is hereby incorporated by reference.

### STATEMENT OF GOVERNMENT SUPPORT

This invention was made with Government support under Grant No. CA168930 awarded by the National Institutes of Health. The Government has certain rights in the invention.

### REFERENCE TO AN ELECTRONIC SEQUENCE LISTING

The contents of the electronic sequence listing (B066270117US01-SUBSEQ-KVC.xml; Size: 62,453 bytes; and Date of Creation: Mar. 20, 2025) is herein incorporated by reference in its entirety.

### BACKGROUND

The IgA nephropathy (IgAN), also known as Berger's Disease, is the most predominant form of primary glomerulonephritis worldwide, accounting for ~30% of the terminal renal failures in patients within 10-20 years after diagnosis. The pathogenesis of IgAN is driven by the mesangial granular deposition of immune complexes (ICs) containing IgA. IgAN demonstrates significant clinical variability arising from the underlying genetic and environmental complexity contributing to the disease pathology. The cause of the majority of primary IgAN cases worldwide are unknown, as the condition is largely sporadic, and only a minority of cases have been reported within clusters of families.

### SUMMARY

Aspects of the present disclosure relate to a method of detecting levels of an antibody in a sample derived from a subject, comprising: contacting the sample with a antigen, wherein the antibody, if present, binds to the antigen; and measuring the amount of antibody bound to the antigen.

Aspects of the present disclosure relate to a method of detecting levels of IgM antibody in a sample derived from a subject, comprising: contacting the sample containing an IgA molecule comprising a GalNAc-α1-Ser/Thr antigen (Tn antigen, also known as CD175), wherein the IgM antibody, if present, binds to the GalNAc-α1-Ser/Thr antigen; and measuring the amount of IgM antibody bound to the IgA molecule.

In some embodiments, the antibody is an IgM antibody that binds a GalNAc-α1-Ser/Thr antigen. In some embodiments, the antigen is an IgA1 comprising the GalNAc-al-Ser/Thr antigen.

In some embodiments, the step of contacting further comprises incubating the sample with the antigen. In some embodiments, the incubating is for a sufficient time for the antigen to bind to the antibody.

In some embodiments, the antigen is adhered to a solid substrate. In some embodiments, the solid substrate is a microbead.

In some embodiments, the sample is blood, blood serum, blood plasma, blood fraction, saliva, mucous, urine, or a combination thereof. In some embodiments, the sample is blood serum.

In some embodiments, the step of measuring comprises the use of flow cytometry to detect the amount of antibody bound to the antigen. In some embodiments, the method further comprises separately contacting a second sample with a control antigen and comparing the amount of antibody bound to the antigen to the amount of antibody bound to the control antigen. In some embodiments, the amount of antibody bound to the antigen being higher than the amount of antibody bound to the control antigen is indicative of a positive result. In some embodiments, the positive result is indicative of a diagnosis of IgA nephropathy or Berger's disease.

In some embodiments, the subject is a mammal. In some embodiments, the subject is a human. In some embodiments, the subject has, is suspected of having, or is at risk of developing IgA nephropathy or Berger's disease.

Aspects of the present disclosure relate to a method, comprising: administering to a subject a glycomimetic, wherein the glycomimetic is based on the structure of N-acetylgalactosamine (GalNAc); wherein: the subject has, is suspected of having, or is at risk of developing IgA nephropathy or Berger's disease.

Aspects of the present disclosure relate to a method, comprising: administering to a subject a glycomimetic, wherein the glycomimetic is α-methylGalNAc or DiαGalNAc; wherein: the subject has, is suspecting of having, or is at risk of developing IgA nephropathy or Berger's disease.

In some embodiments, the glycomimetic binds an antibody that is elevated in subjects with IgA nephropathy or Berger's disease. In some embodiments, the glycomimetic is an αGalNAc monosaccharide or disaccharide. In some embodiments, the glycomimetic is a modified αGalNAc, wherein the αGalNAc is modified to change the —OH group(s) and/or N-acetyl group. In some embodiments, the glycomimetic is an αGalNAc disaccharide. In some embodiments, the αGalNAc disaccharide further comprises linkers linking the two αGalNAc molecules. In some embodiments, the αGalNAc disaccharide comprises a carbohydrate or non-carbohydrate linker. In some embodiments, the linker is a flexible or non-flexible linker. In some embodiments, the linker is a cleavable or stable linker. In some embodiments, the glycomimetic comprises one, two, three, four, five, six, seven, eight, nine, or ten linkers.

In some embodiments, the glycomimetic further comprises one or more constituents on the αGalNAc. In some embodiments, the constituent is a carbohydrate or a non-carbohydrate constituent.

In some embodiments, the glycomimetic is administered orally, subcutaneously, or intravenously. In some embodiments, the glycomimetic is administered as an oral capsule. In some embodiments, the glycomimetic, after administration, is absorbed through the gut.

In some embodiments, the glycomimetic inhibits formation of an immune complex comprising an antibody and an antigen, wherein the antibody is Anti-Tn and the antigen is an IgA comprising a GalNAc-α1-Ser/Thr antigen. In some embodiments, the glycomimetic is capable of dissociating the immune complex. In some embodiments, the glycomimetic inhibits proliferation of mesangial cells.

In some embodiments, the subject is human. In some embodiments, administration of the glycomimetic is a treatment for IgA nephropathy or Berger's disease. In some embodiments, the glycomimetic reduces symptoms of IgA nephropathy or Berger's disease by 5%, 10%, 20%, 30%, 40%, 50%, 60%, 70%, 80%, 90%, 95%, or 100%. In some embodiments, the glycomimetic reduces the risk of developing IgA nephropathy or Berger's disease by 5%, 10%, 20%, 30%, 40%, 50%, 60%, 70%, 80%, 90%, 95%, or 100%. In some embodiments, the method further comprises administering to the subject the glycomimetic and a pharmaceutically acceptable excipient.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

### BRIEF DESCRIPTION OF THE DRAWINGS

The accompanying drawings, which constitute a part of this specification, illustrate several embodiments of the invention and together with the description, provide non-limiting examples of the invention.

FIG. 1 shows basic donor information.

FIG. 2 shows structures on Tn glycopeptide array.

FIGS. 3A-3H show Anti-Tn antibodies in human sera: identification and characterization. FIG. 3A shows Coomassie-stained SDS-PAGE analysis of affinity-purified anti-Tn antibodies from serum using Tn(+)matrix (Elution) and control (beads alone); affinity-purified materials (top) immunoblotted as indicated (bottom). FIG. 3B shows a similar analysis to FIG. 3A (healthy donors, FIG. 1), with purified control antibodies and BSA (4 mg); VVA detects Tn(+)IgA1. FIG. 3C shows purified anti-Tn antibodies bind to Tn(+) matrix; α-methylGalNAc inhibits binding (Alexa Fluor 488-labeled anti-human IgM antibody, top, green). 4× amount of anti-Tn antibodies mixed with Tn(+)matrix (Alexa Fluor 488-labeled anti-human IgG (middle), IgA (bottom)). Beads alone, negative control. DIC=bead images. Performed in 3 biological replicates (C1, C6, C10). FIG. 3D shows a Tn glycopeptide array probed with purified anti-Tn antibodies from 10 healthy control sera (C3, representative), stained with Alexa Fluor 488-labeled anti-human IgM antibody (see FIGS. 9A-9D). Chart ID corresponds to FIG. 2. FIG. 3E and FIG. 3F show data analysis using GLAD, comparing binding preferences between Tn on IgA1 and non-IgA1 peptides, and correlation of binding patterns in C1-C10. VVA binds Tn glycopeptides (positive control), and anti-Tn (murine anti-Tn IgM) differs from C1-C10 binding. FIG. 3G shows purified Tn(+)IgA1 (from CosmcKO cells) and Tn(−)IgA1 (WT) analyzed by SDS-PAGE WB, probed with purified anti-Tn antibodies (bottom), (VVA, middle), and loading control (IgA, top), n=3. FIG. 3H shows a Tn glycopeptide array probed with purified anti-Tn antibody (C1) after preincubation with purified Tn(+)IgA1 or Tn(−)IgA1, stained with Alexa Fluor 647-labeled anti-human IgM. For Tn glycopeptide arrays, error bars represent ±1 SD of four replicates. RFU=relative fluorescence units.

FIGS. 4A-4F show elevated level of anti-Tn antibodies and total IgA1 in IgAN patients. FIG. 4A shows a depiction of anti-Tn IgM detection directly from human sera using Asialo-BSM microbeads, similar to Tn(+)matrix beads in FIGS. 7A-7B, flow cytometry utilizing Alexa Fluor 488-labeled anti-human IgM for signal detection. FIG. 4B depicts a plot of standard curve of purified anti-Tn IgM from human sera, and isotype human IgM serves as a control. FIG. 4C shows Sera with IgAN (P1-P20) or control (C1-C20) analyzed by flow cytometry using Asialo-BSM microbeads. Donor information listed in FIG. 1. Serum IgM analyzed using ELISA. Box plot represents three independent experiments (control, n=20; IgAN, n=20 ▪, male; ◯, female). ***, Student's t test (p=1.3E-06). n.s., not significant (p=0.07). (d) Affinity-purified anti-Tn antibodies of parallel experiments, as in FIG. 3A, from healthy control (C2) and IgAN patient (P1) were loaded as indicated on top of the immunoblots, and immunoblots were probed for IgM, IgG, and IgA antibodies as indicated at right. FT, flow through. FIG. 4E shows a depiction of Tn(+)IgA1 detection from human serum using HPA microbeads by flow cytometry utilizing FITC-labeled anti-human IgA1 for signal detection. FIG. 4F shows Sera with IgAN (P1-P20) or control (C1-C20) were analyzed by flow cytometry using HPA microbeads. Purified Tn(+)IgA1 and Tn(−)IgA1 were used as 100% and 0% respectively. Sera IgA was analyzed using ELISA. Box plots represent three independent experiments (control, n=20; IgAN, n=20 ▪, male; ◯, female). **, Student's t test (p=2.0E-04). n.s., not significant (p=0.10).

FIGS. 5A-5G shows the identification of anti-Tn CICs in IgAN patient and healthy control sera and complexes dissociate with glycomimetic. FIG. 5A shows BN-APAGE analysis of Tn(+)matrix affinity purified anti-Tn antibodies and total purified IgM. Left, native molecular weight markers for approximation of molecular weight. MDa, megadaltons; CICs, circulating immune complexes; P1, P3, P4 represent three patients, control IgM. FIG. 5B shows Tn(+)matrix affinity-purified ReBaGs6 antibody resolved by BN-APAGE system and blotted for murine IgM. FIG. 5C shows a BN-APAGE analysis of affinity-purified anti-Tn antibodies and total purified IgM from three healthy controls (C2, C3, C6), control IgM immunoblotted for IgM. FIG. 5D shows Coomassie-stained SDS-PAGE gel of affinity-purified anti-Tn antibodies from healthy control (C3) and patient (P3); control antibodies and BSA as indicated. Immunoblots (bottom) of same preparation probed as indicated. VVA and IgA blots performed with appropriate controls-purified Tn(+)IgA1 and Tn(−)IgA1 produced from CosmcKO and WT Dakiki cells, respectively. FIG. 5E shows Anti-Tn CICs purified from serum of IgAN patients immunodepleted with anti-IgA or isotype control antibodies; depleted materials analyzed by BN-APAGE-WB probed for IgM. FIG. 5F shows Anti-Tn CICs purified from IgAN patient's serum treated with mock, α-methylGalNAc (αGalNAc), or α-methylGlcNAc (αGlcNAc); samples analyzed by BN-APAGE-WB and probed for IgM and IgA. FIG. 5G shows Tn glycopeptide array probed with anti-Tn antibodies (C3) preincubated with 20 mM α-methylGalNAc or 20 mM α-methylGlcNAc, stained with Alexa Fluor 488-labeled anti-human IgM. Error bars represent ±1 SD of four replicates. RFU=relative fluorescence units. n=3 except blot images from (FIGS. 5A, 5C, 5E), n=2. * represents normal pentameric IgM.

FIGS. 6A-6R show the activity of anti-Tn CICs on HRMCs. FIGS. 6A-6C show HRMC staining-DAPI (nuclear), anti-Tn CICs (green), Vimentin (red); negative control (mixture of isotypes IgM, IgG, IgA). Experiments performed on 3 IgAN patients (P5, P10, Pmix-pooled P1-P10) and 3 healthy control (HC) (C3, C6, Cmix-pooled C1-C10), representative example. FIGS. 6D-6E show surface staining by anti-Tn CICs. HRMCs stained with anti-Tn CICs purified from IgAN (P5, P10, Pmix) or HC (C3, C6, Cmix), probed as indicated. Right plot, the respective MFI for each antibody is plotted against the control. Error bars represent+/−SEM (n=6, two independent experiments of n=3). FIGS. 6F-6H show the MFI from FIGS. 6D-6E plotted (n=6, two independent experiments of n=3), black and red circles=individual cases, bar=average. FIGS. 6I-6L show the proliferation of anti-Tn CICs on starved HRMCs. FIG. 6I shows the unstimulated (0.5% FBS), stimulated (10% FBS); HRMCs stimulated using IgAN serum (FIG. 6J, Pmix) or HC (FIG. 6K, Cmix) where anti-Tn CICs immunodepleted using Tn(+)matrix beads (ID) or mock. Ki-67, cell proliferation marker (green); DAPI, nuclear staining (blue). FIG. 6L shows the quantification of FIGS. 6I-6K, error bars, triplicates measure (3 areas/well); n.s., not significant. FIGS. 6M-6N show the exogenous addition of purified anti-Tn CICs stimulates HRMCs and glycomimetic inhibits proliferation. FIGS. 60-6P show the exogenous addition of anti-Tn CICs (50 ng/100 microliters/well) purified from IgAN patient or HC to Tn(+)matrix depleted sera from IgAN patient, with/without prior treatment of anti-Tn CICs with α-methylGalNAc or α-methylGlcNAc. Scale bars, 20 mm. FIG. 6Q shows the quantification of FIG. 6M-6P, fold change of Ki-67 positive cells normalized to unstimulated. Error bars, triplicate measure (3 areas/well). Representative of 3 biological replicates, IgAN and HC. FIG. 6R shows the purified anti-Tn CICs from three HC and three IgAN patients resolved by SDS-PAGE-WB, probed as indicated (n=3, technical replicates).

FIG. 7A-7C shows the characterization of Tn(+)matrix beads.

FIG. 8 shows the mass spectrometry analysis of the components of the purified anti-Tn antibodies.

FIGS. 9A-9D show the purified anti-Tn antibodies bind to Tn glycoform containing IgA1.

FIGS. 10A-10E shows the establishment of CosmcKO cell line to generate galactose-deficient IgA1 glycoform, Tn(+)IgA1.

FIGS. 11A-11C show immune complexes of the total serum, from both healthy and IgAN patients.

FIGS. 12A-12C show how Glycomimetics-Di αGalNAc specifically inhibits the binding of anti-Tn CICs to IgA1 glycopeptide.

FIGS. 13A-13C show the cell proliferation assay on primary mesangial cells using serum from IgAN patients and healthy controls.

FIGS. 14A-14C show that purified Anti-Tn CICs from both IgAN patient's and healthy control serum do not stimulate HEK293T cells.

FIGS. 15A-15B show the synthesis of GalNAc dimer (Di αGalNAc) and GlcNAc dimer (Di αGlcNAc).

FIG. 16 shows a diagnostic assay for IgA nephropathy.

### DETAILED DESCRIPTION

The underlying pathology of IgA nephropathy (IgAN), the most common glomerulonephritis worldwide, is driven by the deposition of immune complexes containing galactose-deficient IgA1 (Tn(+)IgA1) in the glomerular mesangium. Here it is reported that novel anti-Tn circulating immune complexes (anti-Tn CICs) contain predominantly IgM, representing large macromolecular complexes of ~1.2 MDa to several MDa sizes together with Tn(+)IgA1 and some IgG. Such complexes are significantly elevated in IgAN patient sera, which contains higher levels of complement C3, compared to healthy individuals. Anti-Tn CICs are bioactive and induce specific proliferation of human renal mesangial cells. It has been found that these anti-Tn CICs can be dissociated with small glycomimetic compounds, which mimic the Tn antigen of Tn(+)IgA1, releasing IgA1 from anti-Tn CICs. This glycomimetic compound can also significantly inhibit the proliferative activity of anti-Tn CICs of IgAN patients. These findings could enhance both the diagnosis of IgAN and its treatment, as specific drug treatments are currently unavailable.

A contributing component to IgAN is the unusual nature of the glycosylation of IgA1. Unlike IgA2, IgA1 contains 22 amino acids in a hinge region (HR), comprised primarily of Ser/Thr/Pro residues, in which 3-6 of the 9 Ser/Thr residues may be modified by O-glycans^{11,12}. Examples of these glycans on IgA1 include the Tn antigen GalNAcα1-Ser/Thr (here designated Tn(+)IgA1 and also termed galactose-deficient IgA1 or Gd-IgA1), along with mono- and/or di-sialyl Core 1 structures GalβGalNAcα1-Ser/Thr (here designated Tn(−)IgA1)^{13,14}. Any form of Tn antigen on IgA1 is considered Tn(+) IgA1 irrespective of healthy and IgAN patient serum. Total IgA1 is frequently elevated in sera of patients with IgAN^{8,9,15-19}, and some of those studies indicate that there is an accompanying elevation of the Tn(+)IgA1 glycoform. Interestingly, while circulating IgA1 contains mixtures of these two major glycoforms, Tn(+)IgA1 and Tn(−)IgA1, the relative proportion of these two glycoforms does not appear to be statistically different in patients, but the overall elevation of IgA1 in patients leads to a concomitant rise in both glycoforms^{13,14}.

Earlier it has been observed that individuals infected by certain parasites express IgM antibodies to Tn antigen^{20,21}. Interestingly, the Tn antigen is commonly expressed in the glycoconjugates of many insects and pathogens^{22,23}. This suggested that human exposure to this antigen is perhaps frequent and prompted the exploration of the potential role of such antibodies to the Tn antigen within Tn(+)IgA1 and their potential to form immune complexes with IgA1. Although immune complexes containing the Tn(+)IgA1 glycoform have been proposed to be associated with the IgAN pathology, little is known about the exact nature of such complexes²⁴. The formation of such complexes is a crucial aspect of the currently existing model of the pathogenesis of IgAN, where a multi-hit hypothesis involves autoantibody production and altered expression of Tn(+)IgA1 glycoforms⁸. The emerging picture suggests that the Tn(+)IgA1 glycoform associates with anti-IgA1 specific IgG, IgA, or IgM antibodies to form large macromolecular immune complexes, depositing in the mesangium and ultimately responsible for disease pathology^{8,25-29}.

Despite many years of study on the CICs responsible for IgAN pathogenesis⁹⁻⁴³, much remains to be learned about the nature of such complexes. Here novel anti-Tn CICs of size ranging from ~1.2 MDa to several MDa were identified and purified, and it was demonstrated that these immune complexes consist predominantly of IgM and to a lesser extent Tn(+)IgA1 and IgG. These anti-Tn CICs are significantly elevated in the sera of IgAN patients together with complement C3. Furthermore, these purified anti-Tn CICs specifically stimulate primary human renal mesangial cells. Importantly, Tn antigen glycomimetics can dissociate these anti-Tn CICs to release Tn(+)IgA1, and also inhibit the proliferative nature of the anti-Tn CICs on primary human renal mesangial cells. These insights could have enormous potential for drug development to treat and manage IgAN. A unique assay was also developed to detect these anti-Tn CICs directly from serum, which could potentially be used as a much-needed early diagnostic tool for IgAN.

A key focus of the study was to define the identities, properties, and major components of the anti-Tn CICs in human serum, particularly focused on IgAN. A key advantage of the approach taken was the affinity purification of such complexes using a Tn(+)matrix, which allowed for the identification and quantification of the anti-Tn CICs and their ability to bind the Tn(+)IgA1. In addition, a key aspect of the study was the generation of unique glycoforms of IgA1, i.e., Dakiki-derived materials with or without Cosmc expression representing Tn(−)IgA1 and Tn(+)IgA1, respectively. Both goals were succeeded and the availability of the Tn(+)matrix and Tn(−)IgA1 and Tn(+)IgA1 glycoforms should be helpful in future studies to determine the interactions of IgA1 with autoantibodies.

To explore this concept in more detail, an affinity approach was developed to isolate and explore the nature of the anti-Tn circulating immune complexes (anti-Tn CICs) associated with Tn(+)IgA1 from human sera. Novel anti-Tn CICs of large macromolecular assemblies were identified, predominantly containing IgM together with Tn(+)IgA1 and some level of IgG. The level of such antibodies was measured, where a correlation with IgAN diagnosis was found. More importantly, such anti-Tn CICs can be disrupted by glycomimetics, which block recognition of the Tn antigen, releasing IgA1 from such complexes. Additionally, the glycomimetic compound blocks the proliferative activity of anti-Tn CICs toward primary human renal mesangial cells. These results have significant implications for the diagnosis and potential treatment of IgAN.

It was also observed that IgM, Tn(+)IgA1, and IgG form CICs of several megadaltons size, which contain predominantly IgM. There may be other proteins associated with these large molecular masses, based on BN-APAGE size estimation and Coomassie stained SDS-PAGE bands, while the smaller size CICs of ~1.2 MDa may contain IgM and few IgA1 molecules. It is not unexpected that the anti-Tn CICs are heterogeneous in terms of combinations of IgM, Tn(+)IgA1, and IgG as well as other apparent sub-stoichiometric level of proteins present within the complexes. It can be speculated that IgM directly interacts with the Tn antigen of IgA1 within anti-Tn CICs that have been purified, since both the Tn matrix used for the pull down and IgA1 itself have the Tn antigen, and the IgM does bind to the Tn antigen. However, it is not yet known whether the anti-Tn CICs that were discovered deposit in the kidney and are responsible for IgAN pathogenesis, but the hypothesis is that elevated levels of anti-Tn CICs containing predominantly complement C3 deposit in the kidney and promote IgAN pathogenesis. On the other hand, whether such anti-Tn CICs that have been purified are common in IgAN patients is a very important question that is the subject of future studies.

Although IgAN is characterized by mesangial deposition of IgA1 with IgG and/or IgM², there is a lack of information as to whether there is colocalization of Tn(+)IgA1 with either IgG and/or IgM in all types of IgAN deposits. Due to the biochemical nature of the study with a limited number of patients and their serum samples, expansion of the study to a large cohort of patients was not possible, so it is not clear at this point whether the inclusion of anti-Tn IgM within anti-Tn CICs is a common characteristic in IgAN or only limited to a small number of patients. However, it has been reported that 4-70% of pediatric patients presenting IgAN have mesangial deposits of IgM⁴⁴. Interestingly, such IgM deposition showed a significant association with glomerular crescent, mesangial hypercellularity, segmental sclerosis, and tubular atrophy/intestinal fibrosis⁴⁵. A similar study that focused on IgM identified IgM antibody deposits in the glomerulus, along with a similar distribution of IgA1 in a specimen from an IgAN patient⁴⁶. There are many other studies examining individual antibody levels (IgG, IgM, and IgA1) or some combinations of them, suggesting their deposition in the kidney of the IgAN patients or in a mouse model⁴⁷⁻⁵¹. On the other hand, a recent study using confocal microscopy on frozen kidney-biopsy specimens, along with other biochemical approaches, observed colocalization of IgG and IgA, thus suggesting a potential colocalization between Tn(+)IgA1 with IgG, but that study did not describe potential IgM colocalization⁵². Surprisingly, many studies in the field have focused on IgA1 but not specifically Tn(+)IgA1, which is the predicted antigen, nor has the glycosylation of these antigenic IgA1 glycoforms in deposits been well characterized chemically. Cumulatively, to determine the importance of anti-Tn CICs in IgAN pathogenesis, a longitudinal clinical study focused on a large cohort of human patients designed to study IgM deposition and disease activity should be considered. These are likely to be topics of future studies.

The results demonstrate that the anti-Tn CICs purified from the healthy controls, although present at lower levels in serum, compared to patients, bind to and can stimulate primary human renal mesangial cells to a similar level equivalently to immune complexes purified from IgAN patient when tested under similar concentrations. However, a major difference in this activity that was observed is their relationship upon treatment with glycomimetics. For example, unlike anti-Tn CICs purified from healthy controls, the CICs purified from IgAN patients failed to stimulate human renal mesangial cells when pretreated with α-methylGalNAc that can cause dissociation of the CICs. This indicates possible differences between the immune complexes purified from healthy control and patients in relation to composition, stoichiometry, and specificity within the components. One of the major differences found is the predominant presence of complement C3, but to gain a deeper understanding of the role of C3 within anti-Tn CICs will require further study at the native macromolecular complexes, proteomic, and compositional levels. It is possible that the components of anti-Tn CICs in the sera of IgAN patients may have reorganized towards low affinity immune complexes together with higher accessibility of the glycomimetic. Such future studies will define the components of the complexes and further characterize IgM and Tn(+)IgA1 within the complexes. In addition, the results indicate that the anti-Tn CICs both from IgAN patients and healthy controls binds to human renal mesangial cells preferentially, as well as to their nuclei. Binding to the nuclei is puzzling and will require further studies as to whether the CICs also contain anti-nuclear antibodies or whether the Tn-containing antigens may somewhat reside within cells, as has been proposed by others⁵³. Such a finding might have implications for IgAN pathogenesis.

It could be predicted that increased levels of circulating Tn(+)IgA1 together with the broad exposure of Tn antigens by various sources, for example, microorganisms and parasitic infections during childhood and beyond with organisms expressing the Tn antigen, could lead to the development of anti-Tn antibodies (particularly IgM)^{17,21,25,54}. In addition, some population-based genome-wide association studies (GWAS) have indicated nearly 20 IgAN risk loci⁵⁵; also in a similar study locus 7p21.3 (C1GALT1, T-synthase), the enzyme important in generating extended O-glycans, and locus Xq24 (C1GALT1C1, Cosmc), the specific molecular chaperone for T-synthase are associated with serum Gd-IgA1 levels^{30,32,56,57}. Consistent with these studies, defects in Cosmc and T-synthase, in IgA1-producing B cells has been suggested to be responsible for generating the Tn glycoform⁵⁸⁻⁶¹, but studies in this area are continuings^{7,62-64}. Thus, consistent with the multi-hit hypothesis as to the origin of IgAN, the presence and elicitation of anti-Tn IgM antibodies along with generation of the Tn(+)IgA1 create the background by which IgAN may arise.

The anti-Tn CICs that were purified from human serum show many biochemical and biological characteristics similar to prior characterize CICs and align with evolving model of CICs known to be important in IgAN pathogenesis^{8,9,26,65}. The data unequivocally demonstrate that anti-Tn CICs that were purified contain galactose-deficient IgA1 as the key component together with IgM; these complexes may also contain some IgG. Tn antigen-based glycomimetics treatment followed by blue native gel analysis shows the presence of polymeric IgA1 within the complexes. Interestingly, purified anti-Tn CICs from healthy control can bind to and stimulate proliferation of human renal mesangial cells; such occasional IgA1 deposition may occur as seen in healthy kidney biopsies^{66,67}, the interactions may be different or not sufficient to cause IgAN pathogenesis. For sera from IgAN patients, the results show that anti-Tn CICs are elevated and complement C3 is elevated within the immune complexes compared to anti-Tn CICs found in healthy control sera; such differences may be critical for driving IgAN pathogenesis. However, it is not yet known whether the anti-Tn CICs that were discovered deposit in the kidney and are specifically responsible for IgAN pathogenesis, but the hypothesis is that elevated levels of anti-Tn CICs containing predominantly complement C3 deposit in the kidney and promote IgAN pathogenesis. Establishing the relevance of anti-Tn CICs to IgAN is important, which is part of ongoing work.

Another important aspect of this study is the development of a sensitive assay to detect as well as monitor anti-Tn IgM antibodies in circulation, which has important implications for IgAN diagnosis and as a tool to follow disease progression. An assay has been developed based on the findings that IgM is dominantly present within the anti-Tn CICs, which contain IgM and IgA1 together. Importantly, the results show that IgM within the CICs can be easily detected by secondary anti-IgM antibodies, whereas IgA1 and IgG cannot be directly and easily detected in the native CICs. As such, it can be reasoned that an assay targeted towards detecting IgM bound to the Tn(+)matrix would be more meaningful and sensitive. Accordingly, utilization of this tool allowed for measurement of significant levels of anti-Tn CICs in the sera of IgAN patients as compared to healthy control sera. Many IgAN diagnostic platforms have focused their efforts on detecting primarily IgA1-IgG, complement-C3 and, in very few instances, IgA1-IgM immune complexes. Future studies should take into account the dominant presence of IgM within the CICs together with other antibodies and complement C3.

Despite many years of studies since the original description of the IgAN¹, and progress made for non-specific inhibition of the renin-angiotensin aldosterone system and steroids for patients, there is no specific therapy for this disease⁴⁷. The insights into the carbohydrate-protein interactions within the CICs led us to test a new class of small molecule glycomimetics⁶⁸. It was found that the α-methylGalNAc-based glycomimetics can dissociate the purified anti-Tn CICs into IgA1 and IgM. Importantly, on the other hand, they also prevent the interaction of the purified anti-Tn antibodies with Tn containing IgA1 glycopeptides, suggesting they can potentially block the formation of the CICs. Cumulatively, glycomimetics represent promising candidates to consider for the treatment of IgAN. It has been reported that the properties of CICs (Tn containing IgA1), for example, size, components, and antigen:antibody ratio, influence the nature of the CICs deposited in the glomeruli⁶⁹⁻⁷⁰. Thus, it can be deduced that the anti-Tn CICs that were purified behave similarly, and that treatment of the anti-Tn CICs with α-methylGalNAc-based glycomimetics can dissociate the Tn(+)IgA1 from the complex and reduce kidney deposition. Such glycomimetics may hold great potential for treatment of patients if the compounds are orally administrable and absorbable. Such possibilities are reasonable, as simple sugar compounds are used to treat other diseases, such as the use of oral fucose or mannose supplements to treat congenital disorders of glycosylation⁷¹⁻⁷²⁻⁷⁶. The glycomimetics, if accessible and stable in the circulation, could be effective in disrupting anti-Tn immune complexes.

The present disclosure provides, in some aspects, methods of detecting levels of an analyte, such as an antibody, in a sample derived from a subject who has, who is expected of having, or who is at risk of developing a form of renal disease, such as IgA nephropathy or Berger's disease. The present disclosure also provide, in some aspects, compositions for use in treating a form of renal disease, such as IgA nephropathy or Berger's disease, as well as methods of administering the composition to a subject in need thereof.

### Renal Disease

The present disclosure is related, in part, to the development of diagnostic methods, treatment methods, and therapeutic compositions for a form of renal disease. Renal disease, or kidney disease, is characterized by gradual loss of normal kidney function. Over time, gradual loss of kidney function results in end-stage renal disease (ESRD) or end-stage kidney disease. Treatment for ESRD requires blood dialysis and eventually kidney transplant.

The kidneys function by filtering waste material and excess fluid from the blood. As the kidneys fail and lose normal function, waste builds up in the body and results in a variety symptoms. Examples of symptoms of kidney failure include decreased urinary output, swelling, nausea, fatigue, shortness of breath, and in serious cases, death.

The present disclosure is related, in part, to kidney disease that affects glomeruli. Glomeruli are small networks of blood vessels that are vital to the waste removal function of kidneys. In some embodiments, the present disclosure is related to the buildup of toxic complexes that block glomeruli function. In some embodiments, the toxic complexes are immune complexes that are elevated in individuals who have, who are suspected of having, or who are at risk of developing kidney disease. In some embodiments, the present disclosure is related to detecting levels of toxic immune complexes in a sample derived from a subject who has, who is suspecting of having, or who is at risk of developing kidney disease. In some embodiments, the present disclosure is related to administering a composition that targets toxic immune complexes in subjects who have, who are suspecting of having, or who are at risk of developing kidney disease.

In some embodiments, the present disclosure is related to a form of kidney disease. In some embodiments, the form of kidney disease is any form of kidney disease that results in loss of normal kidney function. In some embodiments, the form of kidney disease is any form of kidney disease that results in loss of glomeruli function. In some embodiments, the present disclosure is related to a form of kidney disease that is characterized by increased levels of toxic immune complexes. In some embodiments, the form of kidney disease is IgA nephropathy. In some embodiments, the form of kidney disease is Berger's disease. In some embodiments, IgA nephropathy is also called Berger's disease. In some embodiments, IgA nephropathy is referred to as “IgAN.”

### Antibodies and Antigens

The present disclosure is related, in part, to the discovery by the inventors of an antibody that is elevated in subjects who have, who are suspected of having, or who are at risk of developing IgAN. In some embodiments, the antibody is an IgM antibody. The IgM antibody is also referred to as immunoglobulin M and is one of several isotypes of antibodies that are produced in vertebrates. As will be understood by the skilled artisan, antibodies function by binding to antigens. Antibodies are also often characterized by the antigens they bind.

The IgM antibody discovered by the inventors was discovered in circulating blood of subjects who have IgAN. In particular, the IgM antibody of the present disclosure was found to bind to the GalNAc-α1-Ser/Thr antigen found in the hinge region of IgA1. The hinge region, as will be understood by the skilled artisan, is a stretch of antibody heavy chain between the Fab and Fc antibody portions. The GalNAc-α1-Ser/Thr antigen, also known as the Tn antigen, is an O-glycan found in the IgA1 hinge region. In some embodiments, IgA1 in the blood serum from subjects who have IgAN has been found to contain aberrant Tn antigen. In some embodiments, IgM, also referred to as Anti-Tn, binds the Tn antigen found in the IgA1 hinge region and forms a toxic Anti-Tn:IgA1 immune complex. In some embodiments, the toxic immune complex forms toxic deposits in the kidneys of subjects with IgAN. In some embodiments, the toxic immune complex forms toxic deposits in the renal glomeruli mesangium of subjects with IgAN.

In some embodiments, subjects with IgAN have increased levels of total IgA1, as compared to subjects without IgAN. In some embodiments, subjects with IgAN have increased levels of IgA1 comprising the Tn antigen in the hinge region, as compared to subjects without IgAN. In some embodiments, subjects with IgAN have increased levels of IgM (or Anti-Tn), as compared to subjects without IgAN. In some embodiments, levels of IgM (Anti-Tn), total IgA1, or Tn antigen in a sample derived from a subject can be indicative of a diagnosis of IgAN in the subject. In some embodiments, therapeutic compositions can be used to dissociate Anti-Tn from IgA1, thereby blocking the formation of or removing toxic immune complexes from the subject.

### Method of Detecting Analytes in a Sample

The present disclosure is related, in part, to a method of detecting levels of an analyte derived from a subject who has, who is suspected of having, or who is at risk of developing a renal disease. The present disclosure is related, in part, to a method of detecting levels of an analyte derived from a subject who has, who is suspected of having, or who is at risk of developing IgAN. In some embodiments, the analyte is a sample derived from a subject. In some embodiments, the analyte or sample is blood, blood serum, blood plasma, blood fraction, mucous, urine, saliva, tears, or any other tissue or fluid found in a subject. In some embodiments, the analyte or sample is blood serum derived from a subject.

In some embodiments, the present disclosure is related to use of a form of the IgA1 molecule that has been engineered to bind Anti-Tn with higher efficiency than naturally-occurring IgA1. In some embodiments, the IgA1 comprises the Tn antigen in more regions than the hinge region alone. In some embodiments, the IgA1 completely expresses Anti-Tn.

In some embodiments, the method of detecting levels of Anti-Tn comprises collecting a sample or analyte from a subject. In some embodiments, the sample is contacted with the IgA1. In some embodiments, after contacting the sample with the IgA1, the sample and IgA1 are incubated. In some embodiments, incubation is for less than 1 minute, at least 1 minute, at least 2 minutes, at least 3 minutes, at least 4 minutes, at least 5 minutes, at least 6 minutes, at least 7 minutes, at least 8 minutes, at least 9 minutes, or at least 10 minutes. In some embodiments, the incubating is for a sufficient time for the antigen to bind to the antibody. In some embodiments, incubation occurs at room temperature. In some embodiments, incubation occurs at ambient temperature. In some embodiments, incubation occurs at 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

In some embodiments, the IgA1 or any control IgA1 is adhered to a substrate. In some embodiments, the substrate is a solid substrate. In some embodiments, the substrate is a gel substrate. In some embodiments, the solid substrate is a plate. In some embodiments, the solid substrate is a microwell. In some embodiments, the solid substrate is a dish. In some embodiments, the solid substrate is a bead. In some embodiments, the solid substrate is a microbead.

In some embodiments, after contact the sample with the IgA1 and incubating, the IgA1 bound to Anti-Tn, if present, is separated from the sample. In some embodiments, the step of separating requires methods that will be known to the skilled artisan. In some embodiments, the step of separating requires protein extraction, depletion of non-bound molecules, or an immunological assay. In some embodiments, the level of Anti-Tn in the sample is measured. In some embodiments, the level of Anti-Tn in the sample is measured by an enzyme-linked immunosorbent assay (ELISA). In some embodiments, the level of Anti-Tn in the sample is measured by flow cytometry. In some embodiments, the level of Anti-Tn in the sample is measured by another method of determining protein concentration.

In some embodiments, the level of Anti-Tn in a sample derived from a subject who has, who is suspected of having, or who is at risk of developing IgAN is compared to a control measurement. In some embodiments, a second sample derived from the subject is contacted with a control IgA1 that does not comprises the Tn antigen. In some embodiments, the sample and the second sample are treated the same way, except the sample is contacted with the IgA1 and the second sample is contacted with the control IgA1. In some embodiments, contacting the second sample with the control IgA1 does not result in Anti-Tn binding to the control IgA1. In some embodiments, contacting the second sample with the control IgA1 results in minimal or negligible Anti-Tn binding to the control IgA1. In some embodiments, contacting the second sample with the control IgA1 results in fewer Anti-Tn molecules binding the control IgA1 than the number of Anti-Tn molecules binding the IgA1. In some embodiments, the level of Anti-Tn bound to the IgA1 is compared to the level of Anti-Tn bound to the control IgA1. In some embodiments, a control sample derived from a subject who does not have IgAN is contacted with the IgA1. In some embodiments, the level of Anti-Tn detected in the sample contacted with the IgA1 is compared to the level of Anti-Tn detected in the control sample. In some embodiments, if the level of Anti-Tn detected in the sample is higher than the level of Anti-Tn detected in the second sample and in the control sample, the subject may be diagnosed with IgAN.

### Glycomimetics

The present disclosure relates, in part, to a method of administering a pharmaceutical composition to a subject in need thereof. In some embodiments, the pharmaceutical composition is a glycomimetic. The term “glycomimetic,” as used herein, refers to a drug-like compound which mimic the structure and function of native carbohydrates. In some embodiments, the glycomimetic is a compound that incorporates N-acetylgalactosamine (GalNAc). In some embodiments, the glycomimetic is a compound that incorporates a GalNAc derivative. In some embodiments, the glycomimetic is a chemical compound based on the structure of GalNAc. In some embodiments, the glycomimetic is able to inhibit formation of the toxic immune complexes comprises of antibodies to GalNAc within the IgA1 molecule. In some embodiments, the glycomimetic is able to block an antibody that binds the Tn antigen within the IgA1 molecule. In some embodiments, the glycomimetic is able to block Anti-Tn from binding the Tn antigen within the IgA1 molecule. In some embodiments, the glycomimetic is able to dissociate a toxic immune complex that has already formed. In some embodiments, the glycomimetic is able to dissociate Anti-Tn from the IgA1 molecule. In some embodiments, the glycomimetic is of a class of glycomimetics that feature the GalNAc structure. In some embodiments, the glycomimetic has biological activity of inhibiting toxic immune complexes in IgAN. In some embodiments, the glycomimetic is α-methylGalNAc. In some embodiments, the glycomimetic is DiαGalNAc. In some embodiments, the glycomimetic is synthesized by combining two αGalNAc molecules to form a dimer.

In some embodiments, the glycomimetic is an αGalNAc monosaccharide or disaccharide. In some embodiments, the glycomimetic is an αGalNAc disaccharide. In some embodiments, the glycomimetic is a modified αGalNAc, wherein the αGalNAc is modified to change the —OH group(s) and/or N-acetyl group. In some embodiments, the αGalNAc disaccharide further comprises linkers linking the two αGalNAc molecules. In some embodiments, the αGalNAc disaccharide comprises a carbohydrate or non-carbohydrate linker. In some embodiments, the linker is a flexible or non-flexible linker. In some embodiments, the linker is a cleavable or stable linker. In some embodiments, the glycomimetic comprises one, two, three, four, five, six, seven, eight, nine, or ten linkers. In some embodiments, the glycomimetic further comprises one or more constituents on the αGalNAc. In some embodiments, the constituent is a carbohydrate or a non-carbohydrate constituent.

In some embodiments, the present disclosure is related to a method of administering to a subject a glycomimetic that features the GalNAc structure. In some embodiments, the present disclosure is related to a method of administering to a subject α-methylGalNAc or a similar molecule. In some embodiments, the present disclosure is related to a method of administering to a subject α-methylGalNAc. In some embodiments, the present disclosure is related to administering to a subject DiαGalNAc or a similar molecule. In some embodiments, the present disclosure is related to administering to a subject DiαGalNAc. In some embodiments, the glycomimetic is administered as a pharmaceutical composition.

In some embodiments, the glycomimetic binds to an antibody that is elevated in subjects who have IgAN. In some embodiments, the glycomimetic binds Anti-Tn. In some embodiments, the glycomimetic is able to dissociate a toxic immune complex. In some embodiments, the glycomimetic inhibits proliferation of mesangial cells. In some embodiments, the glycomimetic is used for treatment of IgAN. In some embodiments, the glycomimetic reduces symptoms of IgAN by 5%, 10%, 20%, 30%, 40%, 50%, 60%, 70%, 80%, 90%, 95%, or 100%. In some embodiments, the glycomimetic reduces the risk of developing IgAN disease by 5%, 10%, 20%, 30%, 40%, 50%, 60%, 70%, 80%, 90%, 95%, or 100%.

### Subjects

A “subject” to which analyte detection or administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In some embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. In some embodiments, the subject is a human who has, is suspected of having, or who is at risk of developing IgAN. In some embodiments, the subject has a genetic risk factor for developing IgAN. In some embodiments, the subject suspects that they are at risk of developing IgAN. In some embodiments, the subject is unaware that they have or are at risk of developing IgAN. In some embodiments, the subject was previously diagnosed with IgAN and are seeking a confirmatory diagnosis. In some embodiments, the subject is experiencing symptoms of kidney disease. In some embodiments, the subject is experiencing early symptoms of kidney disease. In some embodiments, the subject is experiencing symptoms associated with kidney disease.

### Pharmaceutical Compositions and Routes of Administration

The present disclosure is related, in part, to pharmaceutical compositions. Pharmaceutical compositions described herein can be prepared by any method known in the art. An exemplary method include contacting a glycomimetic described herein with a carrier or excipient and one or more additional ingredients, if necessary, then packing the product in a dose.

The term “administer,” “administering,” or “administration” refers to implanting, absorbing, ingesting, injecting, inhaling, or otherwise introducing a compound described herein, or a composition thereof, in or on a subject. The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “pharmaceutically acceptable excipient” or “pharmaceutically acceptable carrier” refers to a pharmacologically inactive material used together with a pharmacologically active material to formulate the pharmaceutical compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. Any one of the compositions provided in the present application may include a pharmaceutically acceptable excipient or carrier.

The present disclosure is related, in part, to administering a pharmaceutical composition to a subject in need thereof. The pharmaceutical compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1. Presence of Anti-Tn Antibodies that are Specific to Tn(+)IgA1 in Normal Human Sera

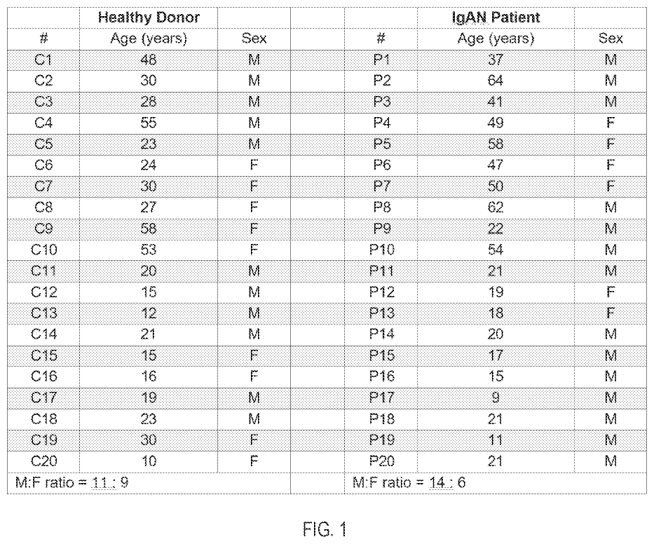

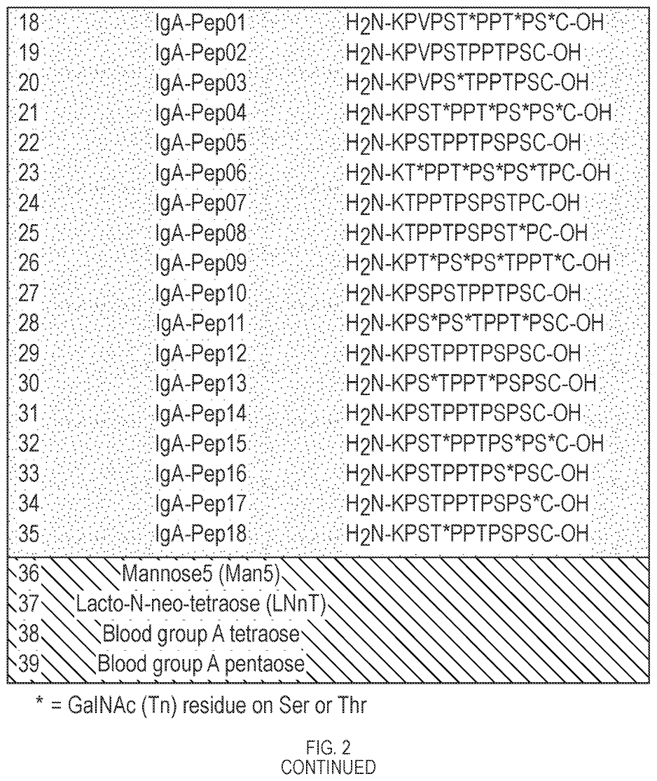

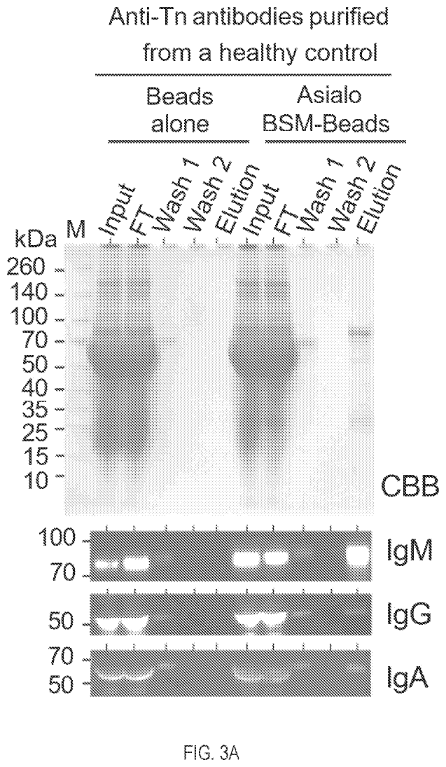

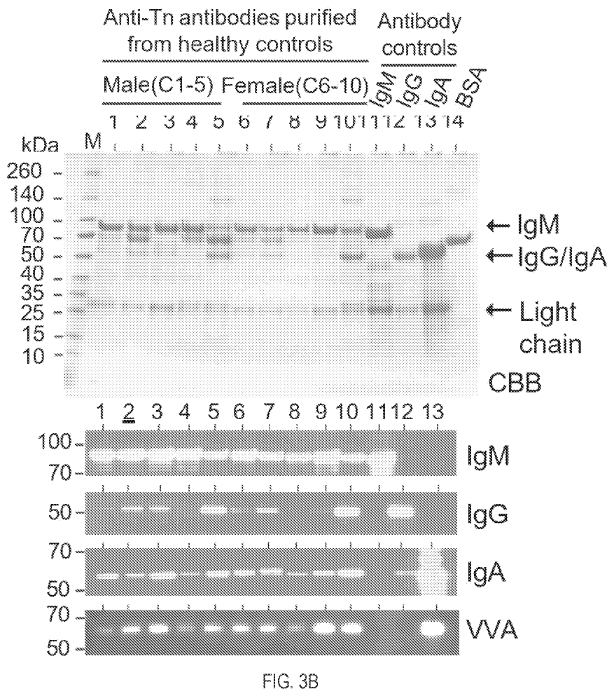

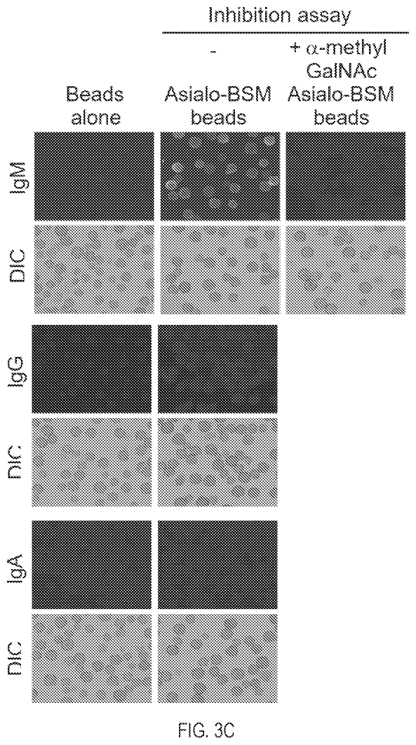

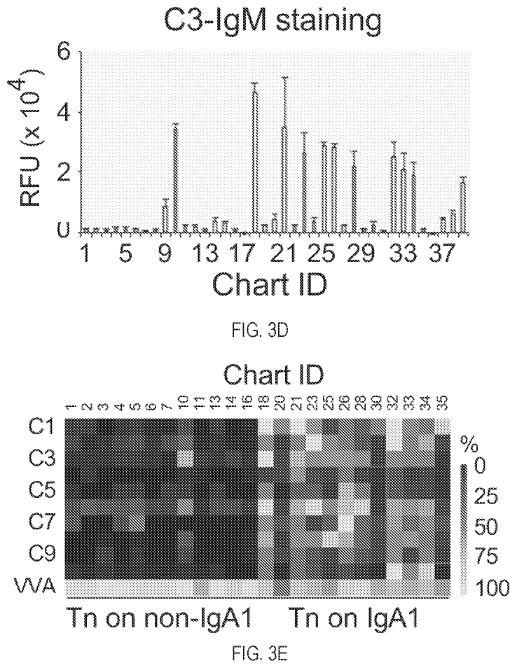

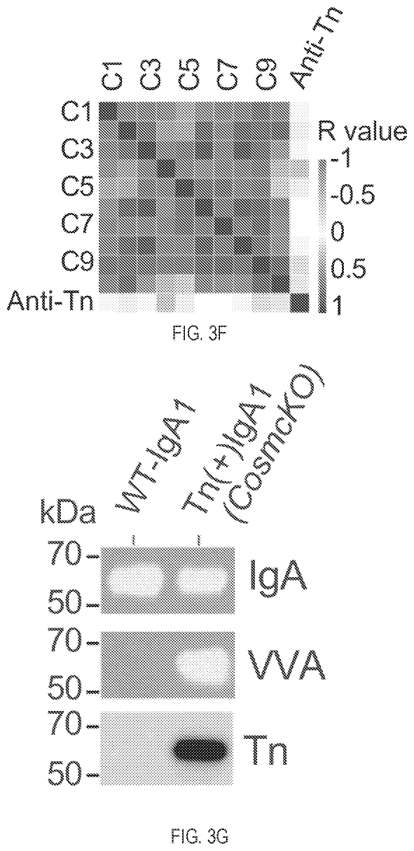

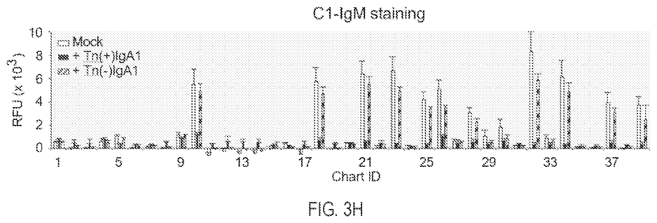

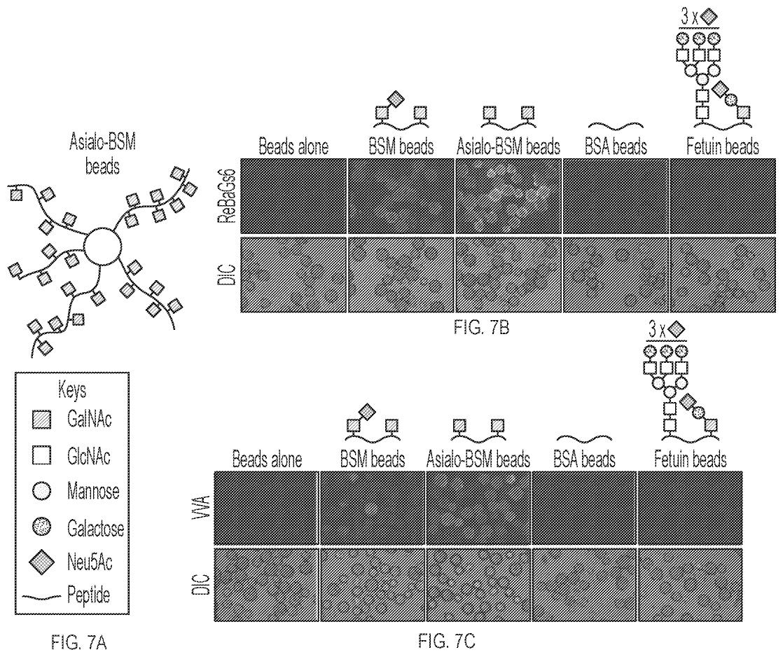

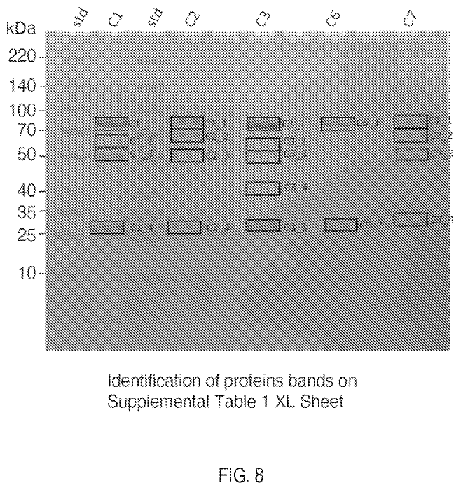

To gain insight into anti-Tn antibodies in human sera and their impact on IgAN pathogenesis, a high density Tn antigen matrix (Tn(+)matrix) was prepared comprising of Asialo-bovine submaxillary mucin (Asialo-BSM), a mucin containing a high amount of Tn antigen along its backbone of primarily Ser, Thr, and Pro residues and potentially mimicking the hinge region of IgA1 in many respects^{17,30} It is possible that the Tn(+)matrix could be used to affinity purify anti-Tn antibodies from human sera (FIG. 7A). As this Tn(+)matrix specifically interacts with a defined anti-Tn antibody and lectin in control studies (FIGS. 7B-7C), the matrix was used to affinity purify anti-Tn antibodies that are potentially present in human sera. The bound materials were resolved on SDS-PAGE, and Coomassie stained. 3-4 major Coomassie stained bands were observed only in the material that bound to the Tn(+)matrix. The protein sizes ranged between ~25 to 75 kDa (FIG. 3A, upper panel, elution lane), suggesting heavy chains and light chains of antibodies. The same preparation was immunoblotted with specific antibodies, which demonstrated that the bound material contains IgM, IgA, and IgG (FIG. 3A, bottom panels). Tn(+)matrix-purified anti-Tn antibodies could be further characterized from the sera of 10 different healthy individuals (FIG. 1), using appropriate controls as indicated on the top of the Coomassie stained gel. These results demonstrate that the purified anti-Tn antibodies from each sample are predominantly IgM (75 kDa), along with lesser amounts and varying proportions of IgG (50 kDa) and/or IgA (55 kDa) (FIG. 3B). Immunoblots confirmed that IgM is present in all samples along with varying amounts of IgG; the Tn(+)IgA1 glycoform was also present in all samples (FIG. 3B, bottom panels). To further confirm that the identified proteins are immunoglobulins (FIG. 3B), a proteomic analyses by mass spectrometry was performed on individual sera samples from 5 controls (C1, C2, C3, C6, and C7). The results demonstrate that the Coomassie-stained bands represent IgM, IgG, and IgA, as expected (FIG. 8). The results of proteomic analyses demonstrate that additional proteins are present, including the consistent presence of complement C3.

To investigate whether the interaction of the anti-Tn antibodies to the Tn(+)matrix is Tn antigen-dependent, fluorescent-tagged secondary antibodies were used to image the antibodies bound to the Tn(+)matrix. Strong staining for bound IgM (FIG. 3C, upper panel, green fluorescent) was observed, but no significant staining for either IgG or IgA (FIG. 3C, middle and bottom panels). The IgG and IgA1 levels in the bound material are thus either relatively low or these antigens could be cryptic within complexes and inaccessible in the context of the beads. This approach was used to determine whether binding of antibodies to Tn(+)matrix can be inhibited by a hapten sugar α-methylGalNAc (a glycomimetic derived from the Tn antigen). Binding of antibodies to the Tn(+)matrix was completely inhibited by α-methylGalNAc pretreatment (FIG. 3C, right most upper panel), thus demonstrating that the interaction is Tn antigen-dependent. These results demonstrate that healthy individuals contain low levels of anti-Tn antibodies that specifically recognize the Tn antigen in a hapten-inhibitable fashion.

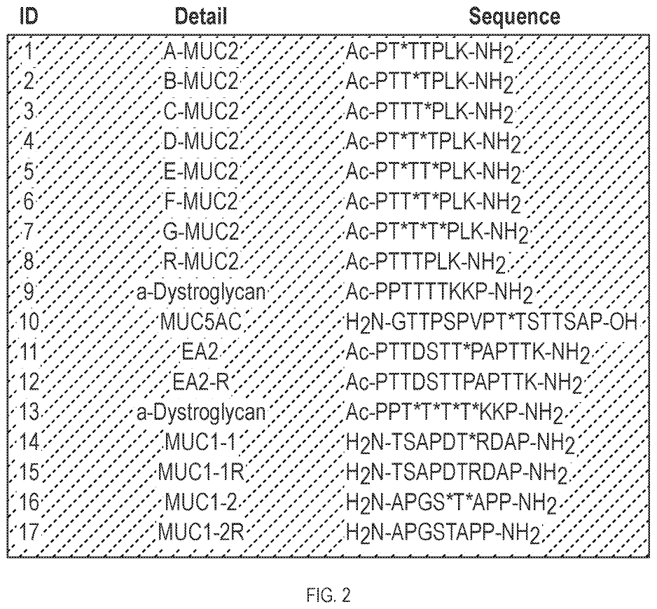

To examine the specificity of antibodies to the Tn antigen, a synthetic Tn glycopeptide array was prepared as a first approach, where small glycopeptides contain the Tn antigen in the context of IgA1 hinge region (Tn(+)IgA1 glycopeptides), or within non-IgA1 peptides as listed (FIG. 2). Affinity-purified antibodies were applied from sera of 10 healthy individuals and detected for the presence of bound IgM.

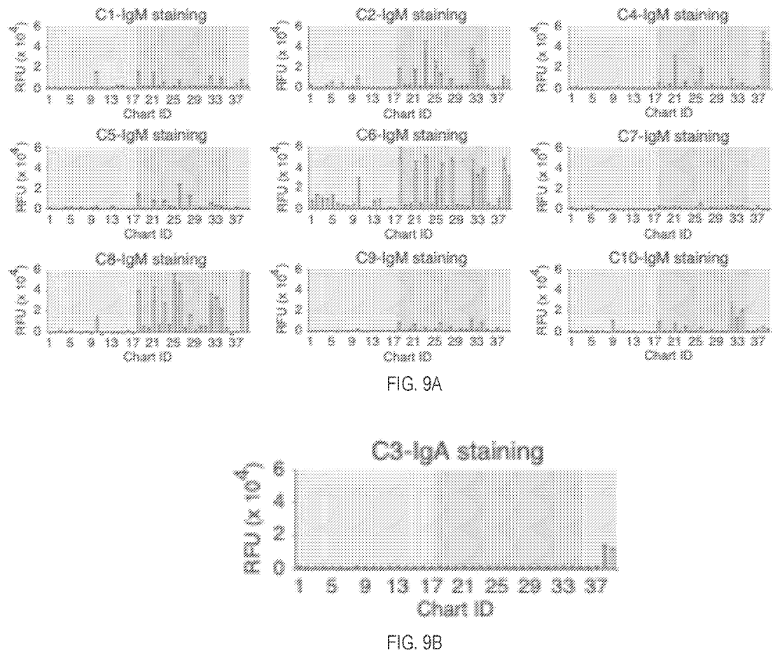

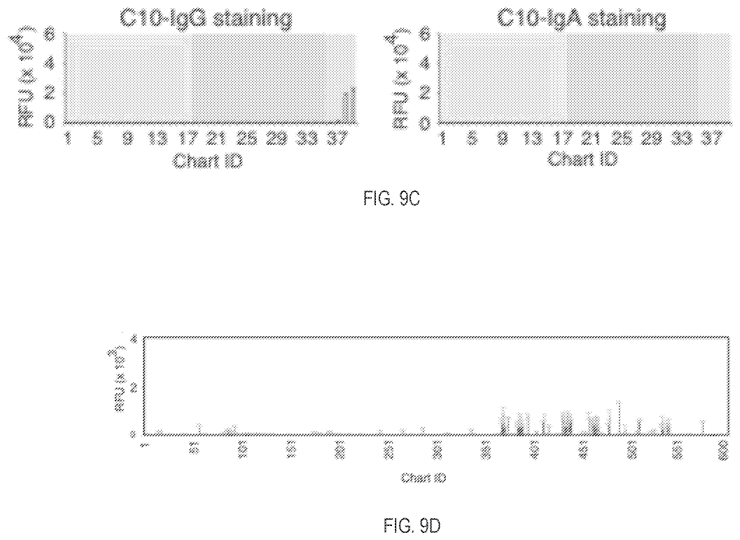

The results demonstrate higher interaction of such IgM with Tn(+)IgA1 glycopeptides compared to other Tn(+)non-IgA1 glycopeptides (FIGS. 3A-3F, FIG. 9A). In parallel experiments, the preparation of this glycopeptide array was also probed for the possible binding of IgA1 and IgG using amounts that were increased four-fold as compared to IgM detection. Signals were not detected above background (FIGS. 9B-9C). Thus, these results indicate that either IgA1 or IgG do not bind the Tn(+)IgA1 glycopeptides or such antibodies are not available within potential complexes, whereas IgM is available and accessible. Additionally, the binding of antibodies to the CFG mammalian glycan microarray was tested—this array contains some glycans expressing terminal GalNAc, but lacks any glycopeptides or sequences resembling the Tn(+)IgA1 glycopeptides. No binding to any glycans with terminal GalNAc was observed, which suggests that the anti-Tn antibodies do not bind to glycans simply expressing terminal GalNAc (FIG. 9D). These data indicate that anti-Tn antibodies in sera are predominantly of IgM subclass which is consistent with FIG. 3B, and that these antibodies bind to modeled glycopeptides based on Tn(+)IgA1.

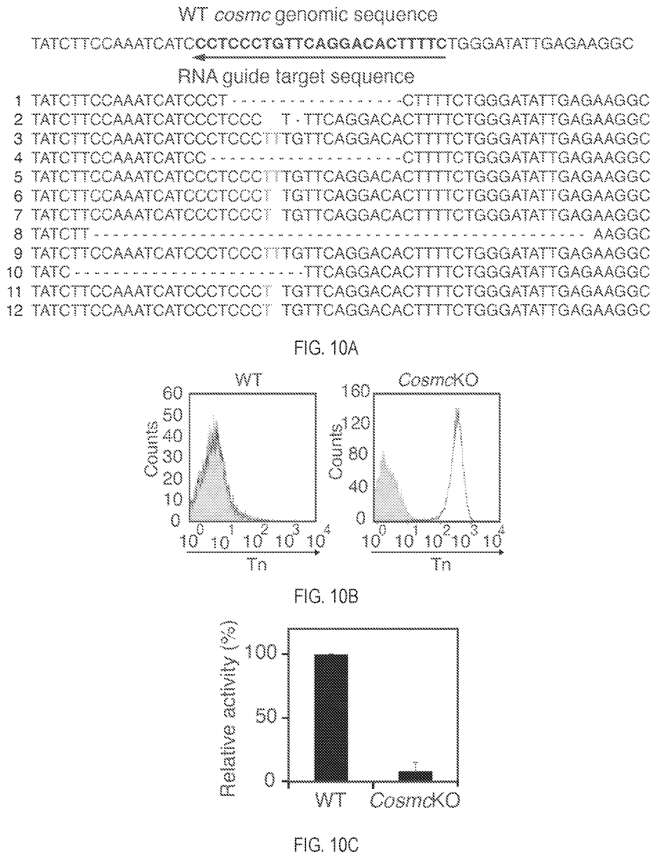

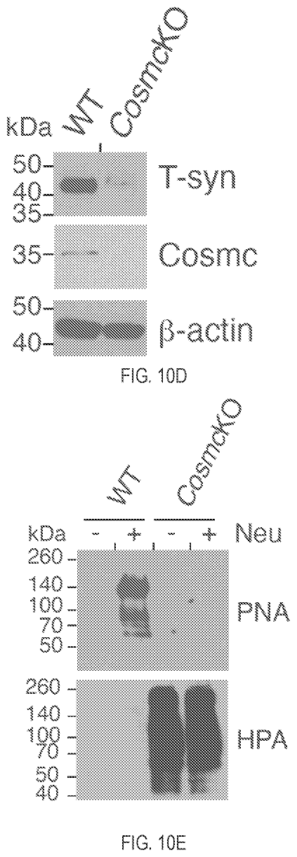

In addition, to more directly demonstrate specific binding of anti-Tn antibodies to Tn(+)IgA1, the fact that the human Dakiki B cell line secretes IgA1¹⁷ was taken advantage of. Using the CRISPR-Cas9 system, the X-linked Cosmc (C1GalT1C1) gene was deleted, thus creating a CosmcKO B cell line that is unable to generate complex O-glycans and can only synthesize the Tn antigen on IgA1 and all other glycoproteins (FIGS. 10A-10E). Cosmc encodes the key molecular chaperone that regulates formation of active T-synthase, required for generating core 1 O-glycans and all further extended forms³⁰⁻³³. The secreted IgA1 was purified from both WT Dakiki cells and CosmcKO B cells. SDS-PAGE immunoblot analysis of the purified IgA1 shows the KO cell line produces the Tn(+)IgA1 glycoform, which is recognizable by the lectin VVA and anti-Tn antibodies from human sera. By contrast, normal WT-IgA1 (Tn(−)IgA1 glycoform), lacks the Tn antigen and is not bound by reagents that bind the Tn antigen (FIG. 3G). It was then questioned whether Tn(+)IgA1 could inhibit binding of affinity-purified anti-Tn antibodies to the Tn glycopeptide array, as observed in FIGS. 3D and 3E. These results demonstrate that Tn(+)IgA1, but not WT-IgA1, can inhibit the binding of the anti-Tn antibodies to the Tn glycopeptide array (FIG. 3H). Together, the results demonstrate that anti-Tn IgM antibodies can specifically interact with Tn(+)IgA1.

### Example 2: Elevated Level of Anti-Tn Antibodies in Sera of Patients with IgAN

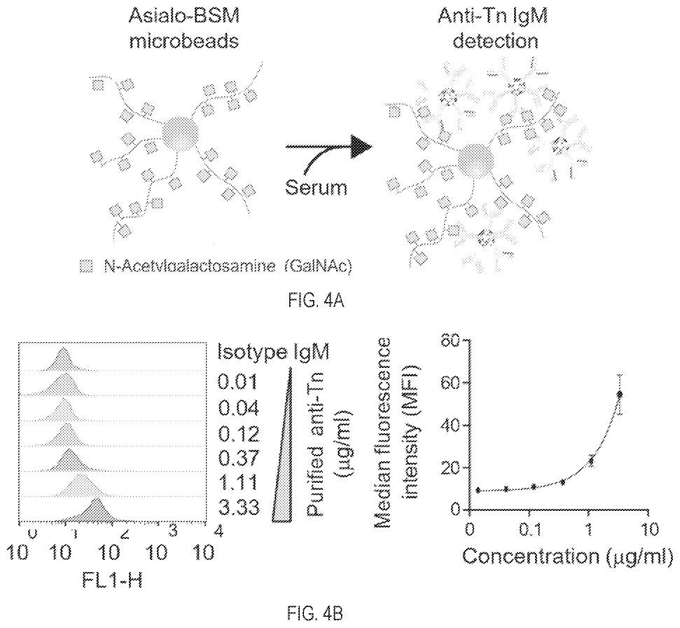

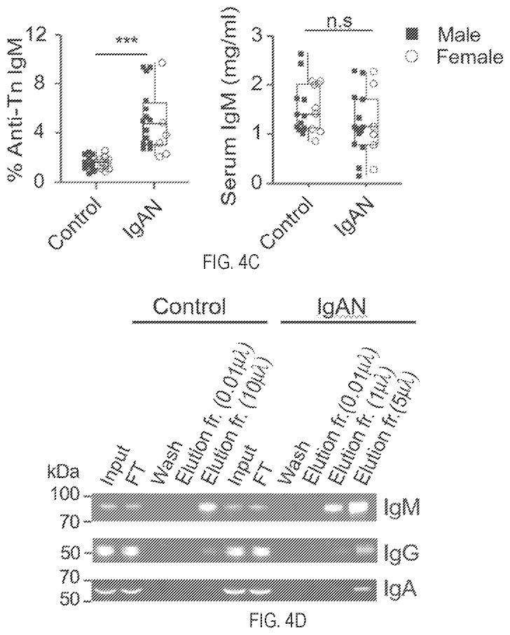

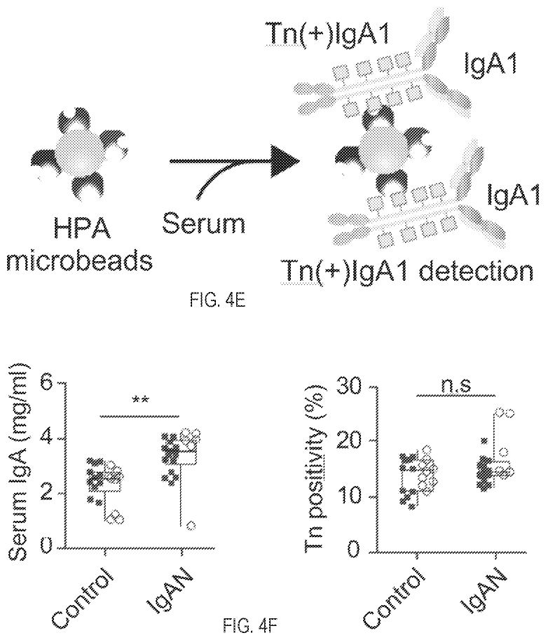

With these tools in hand, the level of anti-Tn antibodies in sera from patients with IgAN was measured and compared to healthy controls. Since affinity-purified anti-Tn antibodies contain predominantly the IgM subclass (FIG. 3B), measurements were focused on the levels of anti-Tn IgM. A highly sensitive flow cytometric assay that utilizes high density conjugation of Asialo-BSM to microbeads was established (FIG. 4A). A standard titration curve using purified anti-Tn IgM from healthy sera was created (FIG. 4B). Using this curve, a relatively high level of anti-Tn IgM in serum from patients with IgAN (2.2-10.6% of total IgM) was measured, as compared to control sera (0.8-2.7% of total IgM). There were no significant differences in total serum IgM in control versus IgAN samples (FIG. 4C). SDS-PAGE immunoblots probed for IgM, IgG, and IgA of the purified anti-Tn antibodies showed consistent results, demonstrating elevations of IgM to the Tn antigen (FIG. 4D). Additionally, the titer of serum IgA1 levels and its percentage of Tn positivity using HPA-microbeads was measured (FIG. 4E). Serum IgA1 levels were elevated in IgAN patients as compared to healthy controls as expected (FIG. 4F, left), but the percentage of Tn positivity of IgA1 in serum did not change in both groups (FIG. 4F, right), suggesting an increased amounts of Tn(+)IgA1 in the circulation of the IgAN patient. These data indicate that the levels of both anti-Tn antibodies and Tn(+)IgA1 tend to be higher in sera of IgAN patients compared to control sera, thus potentially contributing to enhanced formation of IgM and IgA1 immune complexes.

### Example 3: Characterization of Circulating Anti-Tn Macromolecular Immune Complexes (Anti-Tn CICs) Purified from Human Sera

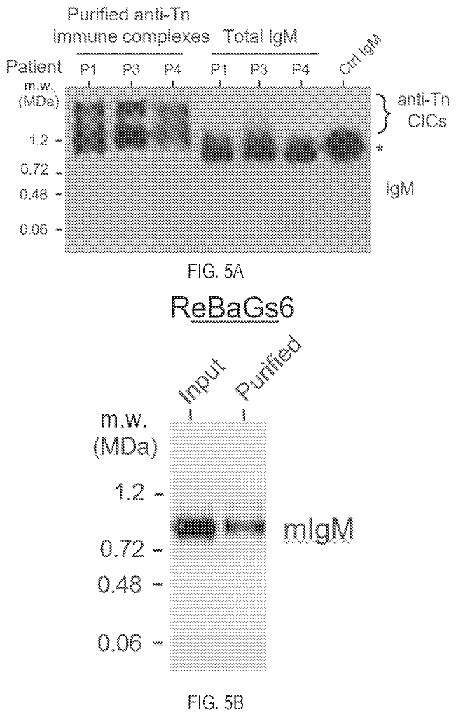

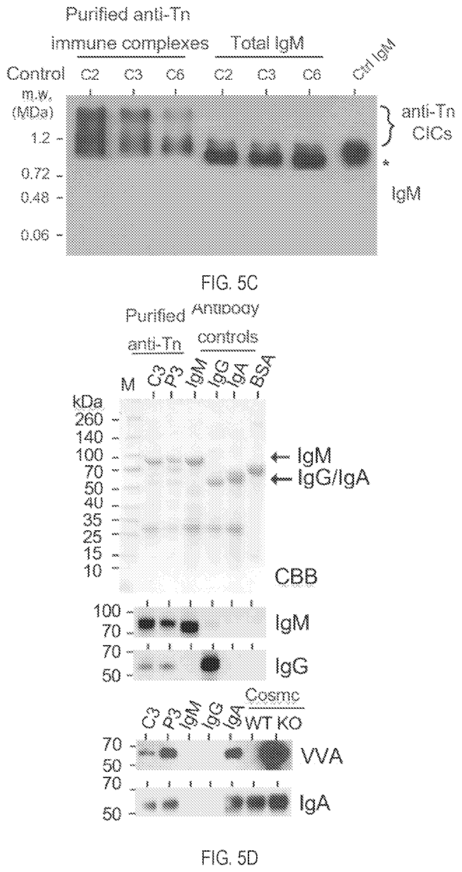

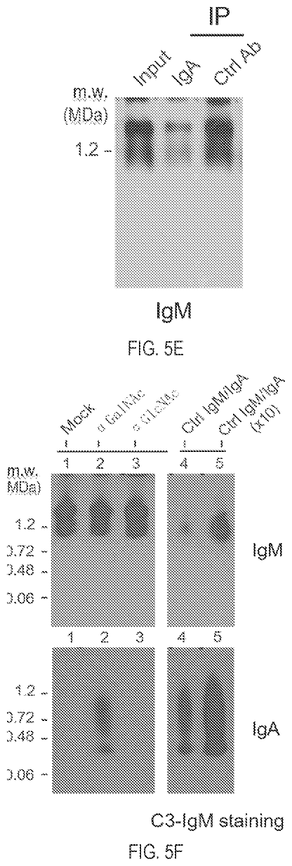

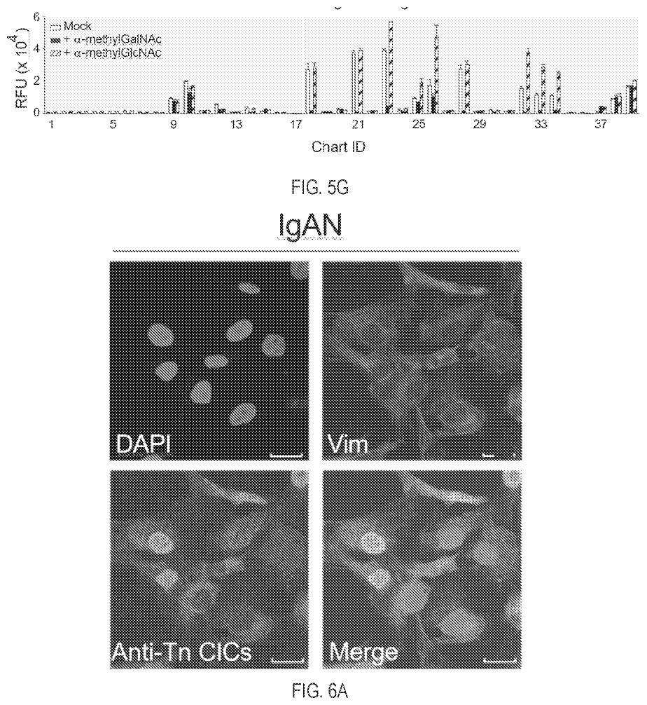

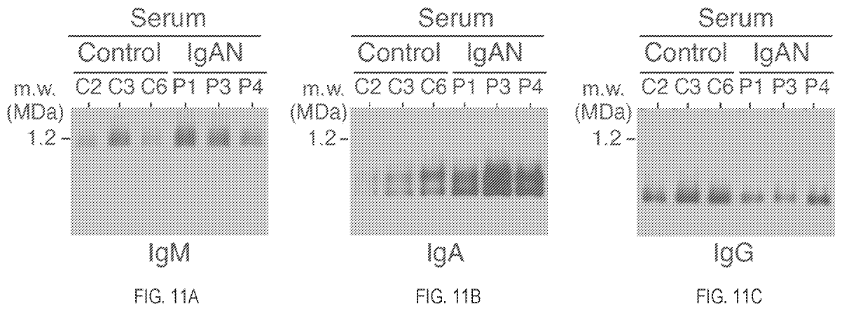

The above results collectively suggest the possibility of circulating immune complexes (CICs) comprised of IgM and Tn(+)IgA1 (FIGS. 3B, 3D-H, and 4D). To explore this possibility, d a blue native-agarose polyacrylamide gel electrophoresis (BN-APAGE) system, which resolves native protein complexes up to 6 MDa or more³⁴. First, using the Tn(+)matrix that was affinity-purified anti-Tn antibodies from control sera and sera from patients (P1, P3, and P4), and used the BN-APAGE system to resolve the bound materials; the resulting immunoblots were then probed for IgM. The results revealed that IgM-containing anti-Tn immune complexes migrated predominantly as large molecular weight species of 1.2 MDa and above, with significantly higher molecular weight species (several megadaltons) than control IgM, which migrated at the expected size of ~0.9 MDa (FIG. 5A). As a control IgM, ReBaGs6 was used, which is an anti-Tn mouse IgM that binds to Tn(+)matrix³⁵. This control antibody was purified using a similar approach on the Tn(+)matrix; the bound ReBaGs6 IgM had a predicted normal size when resolved on BN-APAGE, demonstrating that no artificial complexes are formed by possible leakage of Asialo-BSM from the Tn(+)matrix (FIG. 5B). In parallel, BN-APAGE was used to analyze the sizes of affinity-purified anti-Tn antibodies as well as total IgM (similar concentration used as in FIG. 5A) from controls (C2, C3, and C6). These CICs also showed similar behavior of anti-Tn IgM immune complexes from patients (FIG. 5C), indicating that the immune complexes from both healthy controls and patients appear to be similar at least in terms of apparent mass but differ as shown above in total levels, being higher in patient sera. No anti-Tn IgM antibody was observed as being similar to the size of pentameric IgM in both healthy and IgAN patients (FIGS. 5A and 5C), suggesting that the anti-Tn IgM is associated with other molecules, possibly IgA1 and/or IgG, as these were observed also in the original Tn(+)matrix bound materials (FIG. 3B), along with possibly other serum glycoproteins. In similar analyses of the purified anti-Tn CICs from IgAN patients, as defined in FIG. 3B, 3-4 distinguishable bands in IgAN patients were observed potentially representing IgM, IgG, and Tn(+)IgA1 (FIG. 5A). As controls, total sera from both healthy control and IgAN cases was analyzed using BN-APAGE system, probed for total IgM, IgG, and IgA, and did not observe higher molecular weight complexes under these conditions, which suggests that CICs are a relatively small component of overall sera (FIG. 11A-11C). Together, these data indicate that CICs of megadalton size could potentially contain all of the components that account for complexes in the megadalton mass. However, even the smallest CICs would be predicted to contain at least IgM and one or more of IgA and/or IgG.

### Example 4: Anti-Tn CICs Contain IgM and IgA1 and the Tn Antigen-Based Glycomimetics Disrupt CICs

The above results do not exclude the possibility that Tn(+)IgA1 could be confined only to one complex or may not be present at all within the anti-Tn IgM complexes. To examine the distribution of the Tn(+)IgA1 within the large anti-Tn CICs containing IgM (FIGS. 5A, 5C, and 5D), the anti-Tn CICs were immunodepleted using anti-IgA and control antibodies and observed specific co-depletion of IgM with IgA (FIG. 5E, middle lane). This result is consistent with the prediction that IgM and IgA are associated with each other within the complexes.

Furthermore, it was tested whether the anti-Tn IgM immune complexes can be dissociated from Tn(+)IgA1 using compounds that mimic the Tn antigen-glycomimetics. For this purpose, the anti-Tn CICs were affinity-purified from the sera of IgAN patients; the anti-Tn CICs were treated with mock, α-methylGalNAc (glycomimetic compound, αGalNAc), or α-methylGlcNAc (control compound, αGlcNAc), followed by BN-APAGE-WB and probing for IgM, and IgA. Interestingly only α-methylGalNAc, but not the control α-methylGlcNAc, caused dissociation of the Tn(+)IgA1 from anti-Tn IgM immune complexes (FIG. 5F, lane 2, bottom), releasing Tn(+)IgA1, which had a similar electrophoretic mobility to control native IgA1 (FIG. 5F lanes 4 and 5, bottom). An IgA1 signal was unable to be detected in mock and α-methylGlcNAc-treated samples (FIG. 5F, lane 1 and 3, bottom), indicating the IgA1 epitopes may be buried within the CICs and thus not available in this native page set-up for WB detection.

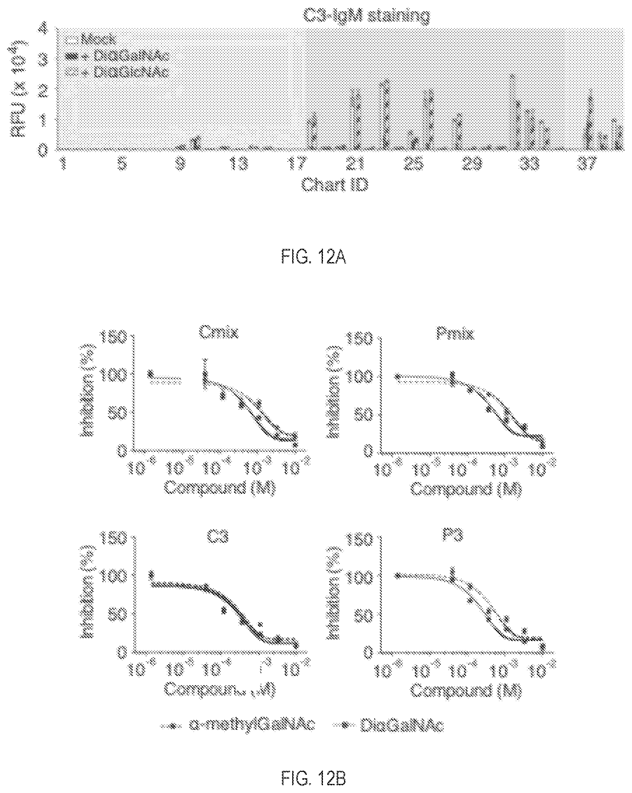

Next, the efficacy of α-methylGalNAc to block binding of anti-Tn IgM was analyzed, which could be used to prevent the formation of the circulating macromolecular anti-Tn CICs, since anti-Tn IgM complexes have available Tn antigens sites and the IgM within binds to both Tn-containing IgA1 glycopeptides and intact IgA1 (FIGS. 3D-3H). Upon analysis using the Tn glycopeptide array, as in FIG. 3A-3H, α-methylGalNAc specifically inhibited binding of the anti-Tn IgM (FIG. 5G). A dimeric version of the Tn antigen, termed DiαGalNAc (i.e., GalNAc dimer) along with a control DiαGlcNAc (i.e., GlcNAc dimer) was further synthesized. The DiαGalNAc exhibited higher inhibition compared to α-methylGalNAc in terms of anti-Tn antibody binding to the array, as listed (FIG. 2, FIG. 12A). Furthermore, the IC50s were calculated using an ELISA based approach, where Tn glycopeptides were used from IgA1 (ID 18 and 19, listed in FIG. 2), and performed the inhibition experiments using the glycomimetics (i.e. α-methylGalNAc and DiαGalNAc). A higher inhibition of anti-Tn CICs binding with DiαGalNAc treatment was observed (FIGS. 12B-12C). Together, the result indicates that the anti-Tn CICs are composed of at least IgM and IgA, and Tn antigen glycomimetics specifically dissociate Tn(+)IgA1 from the complexes.

### Example 5: Glycomimetic Treatment Inhibits the Proliferative Nature of Anti-Tn CICs on Primary Human Mesangial Cells

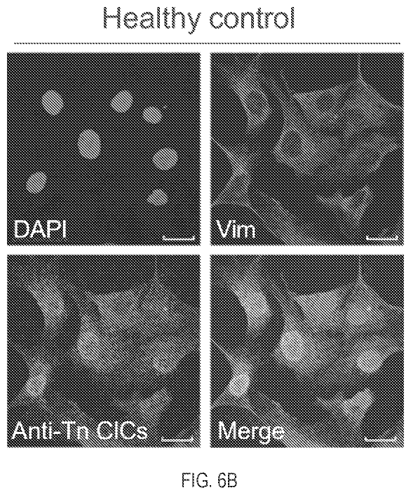

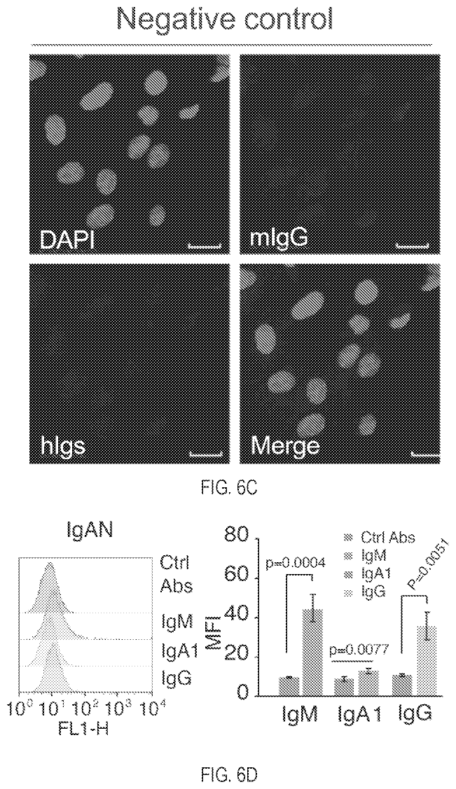

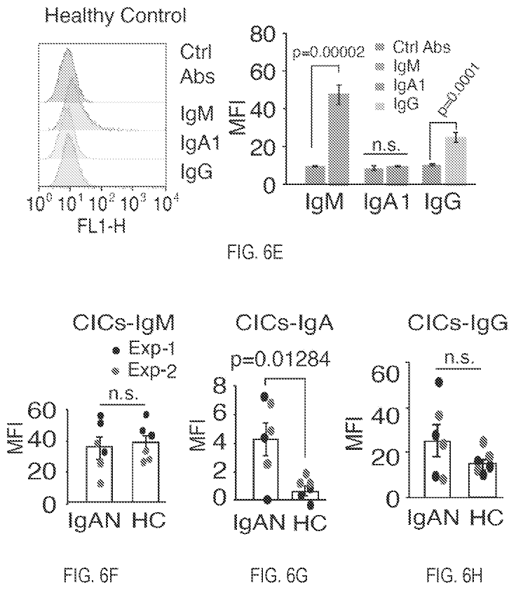

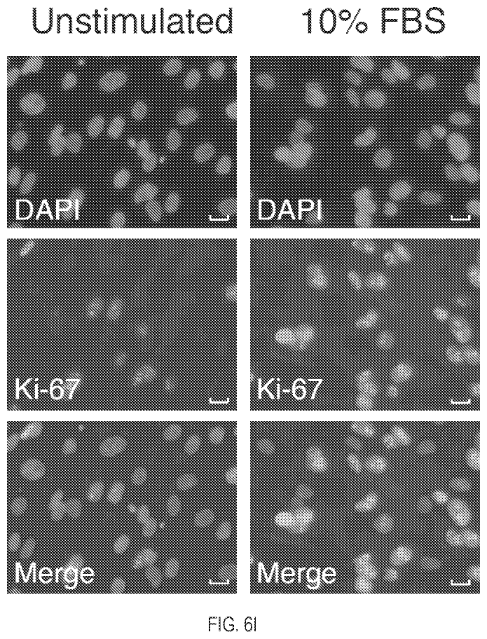

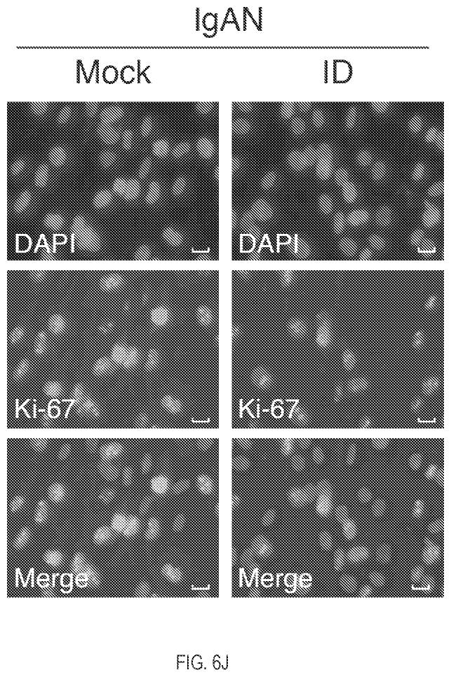

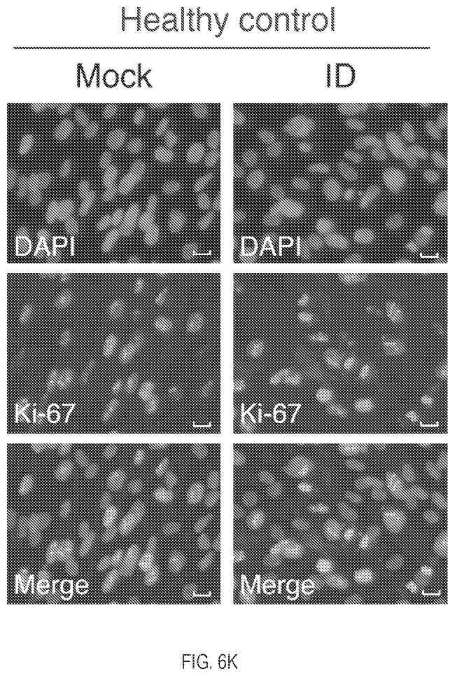

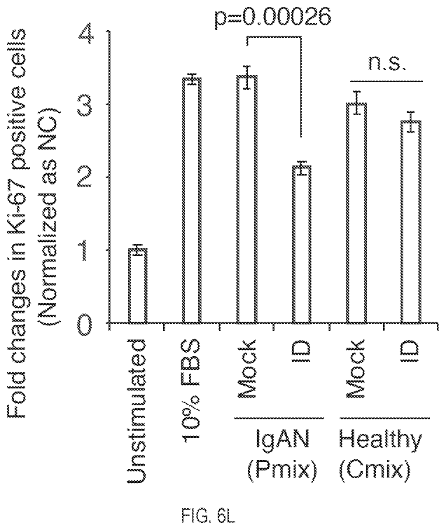

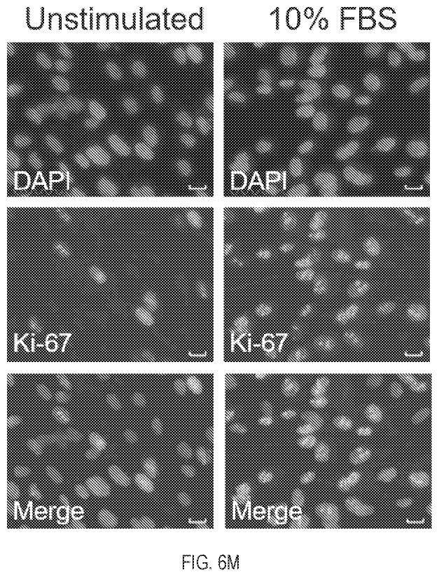

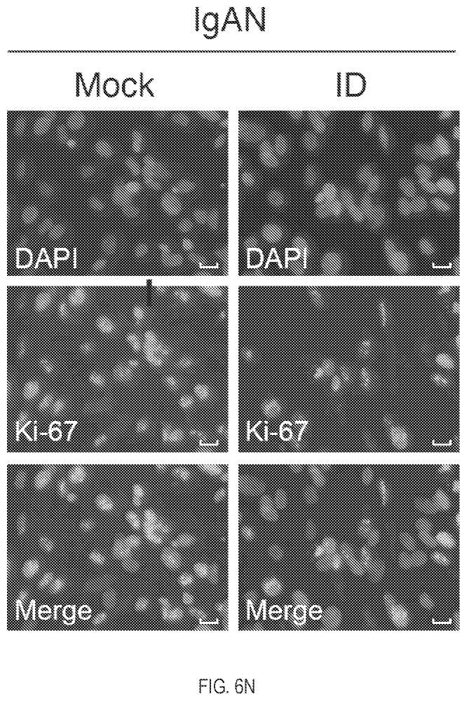

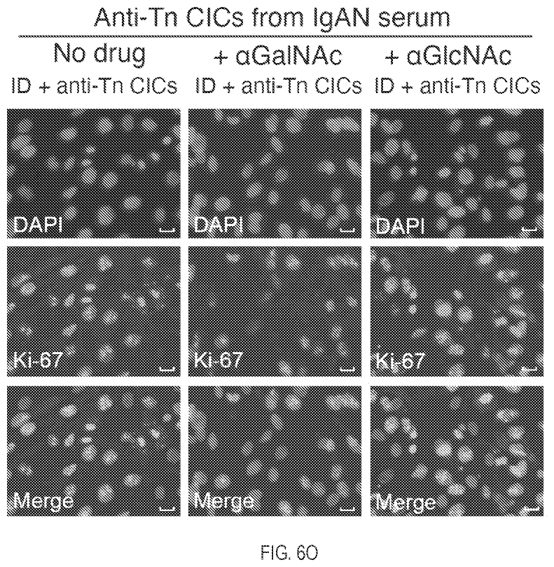

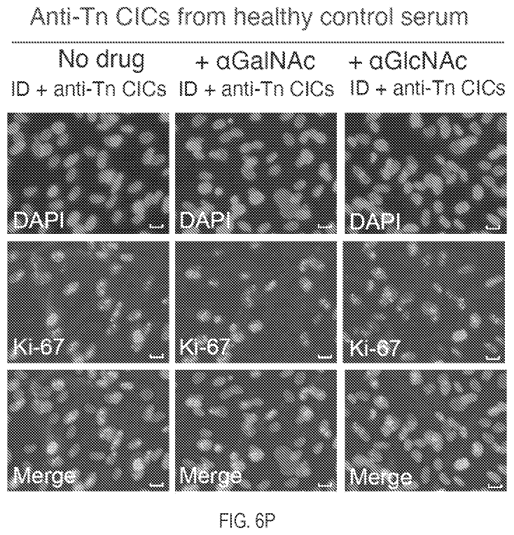

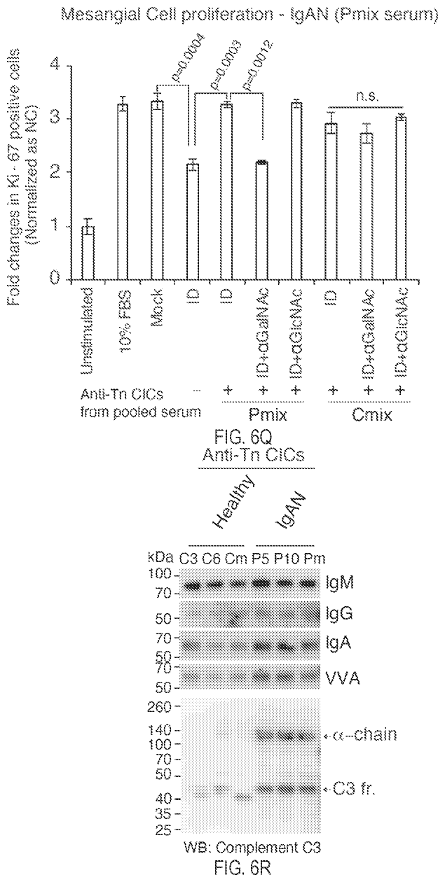

Several studies suggest that immune complexes containing Tn(+)IgA1 from IgAN patients stimulate mesangial cell proliferation³⁶⁻³⁸. It was therefore analyzed whether the purified anti-Tn CICs have bioactivity toward mesangial cells. First, the binding nature of anti-Tn CICs to mesangial cells was analyzed. For this, the cultured cells were fixed, permeabilized with 0.05% Triton-X-100, and stained with anti-Tn CICs. It was found that anti-Tn CICs from both IgAN patient and healthy control bind to the cells, whereas isotype control antibodies do not bind (FIGS. 6A-6C); this is consistent with the characteristics of autoantibodies. Furthermore, anti-Tn CICs binding to mesangial cell surfaces were also analyzed, using flow cytometric analysis; next the cells were stained with anti-Tn CICs and analyzed the binding, and it was observed significant binding not only with the predominant component of anti-Tn CICs (IgM), but also with IgA1, and IgG (FIGS. 6D-6E) to mesangial cells (except IgA1 in the case of healthy control) indicating that anti-Tn CICs can interact with the mesangial cell surface. This led to a question regarding whether there is a difference in anti-Tn CICs, purified from IgAN and healthy control serum, binding to mesangial cells, and the analysis showed that there is no difference in binding except in the case of IgA1 (FIGS. 6F-6H).

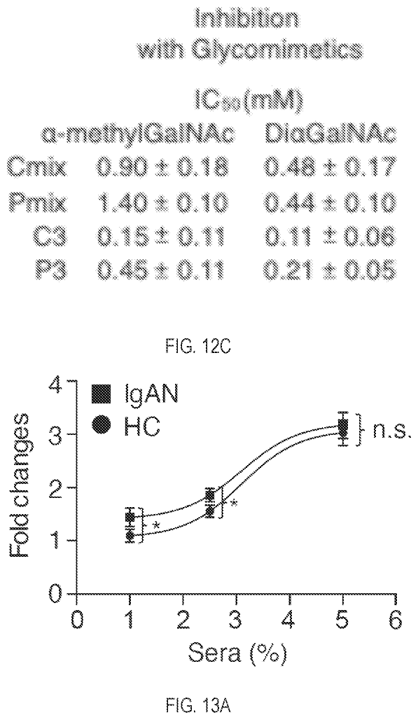

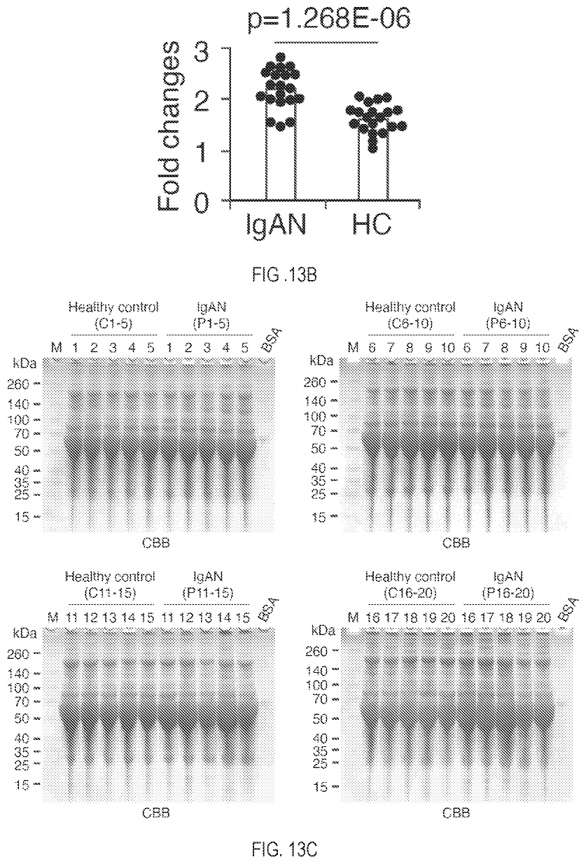

To investigate the proliferative nature of the anti-Tn CICs, sera was directly utilized from both IgAN patients and healthy controls to stimulate primary mesangial cells, which were cultured under 0.5% FBS-containing media, and directly stimulated the cells with different concentrations of the serum, then stained for the proliferation marker Ki-67. Results using stained cells were calculated as a fold-change. It was found that both sources of sera stimulate mesangial cells in a dose-dependent manner, but stimulation by sera from IgAN patients was significantly higher than healthy controls, and the difference was more evident in lower serum concentrations (1 and 2.5%); at 5% the difference was not significant, perhaps due to saturation (FIGS. 13A-13B). There were no obvious changes observed in total serum protein with IgAN compared to healthy controls (FIG. 13C). Next, anti-Tn CICs were immunodepleted with the Tn(+)matrix or mock using the sera from both IgAN and healthy control; the immunodepleted sera were added to cells, and stained for Ki-67. The results demonstrate that the depletion of anti-Tn CICs from IgAN patient sera significantly reduces its proliferative nature of the mesangial cells compared to mock depletion (FIGS. 6I, 6J, 6L, Mock and ID, Pmix), indicating that anti-Tn CICs are bioactive. In parallel experiments with healthy control sera, no significant changes were observed in proliferative nature of the sera depleted of anti-Tn CICs (FIGS. 6I, 6K and 6L, Mock and ID, Cmix), which may also be due to the significantly lower amount of anti-Tn CICs and potential differences in the components of the CICs in the healthy control sera as compared to IgAN patient (FIG. 4C).

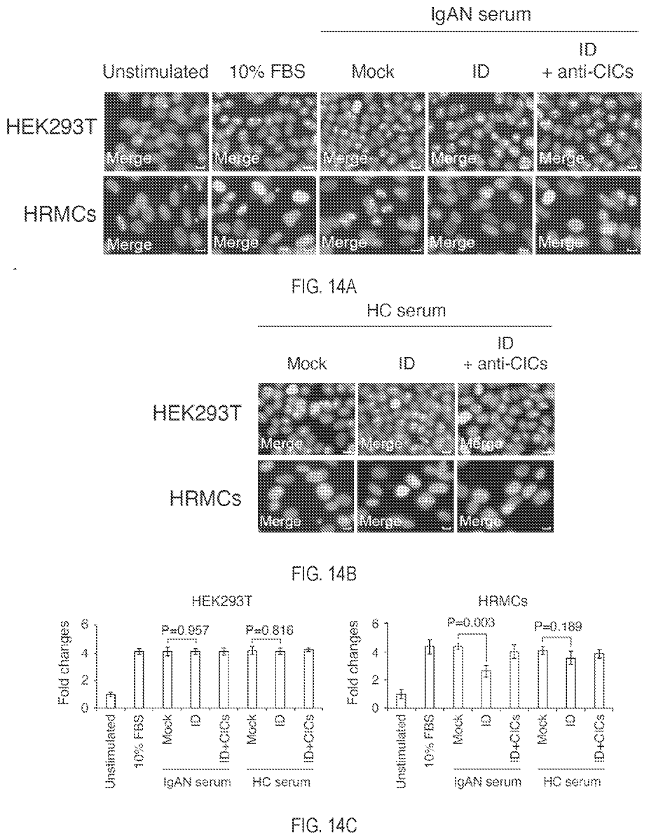

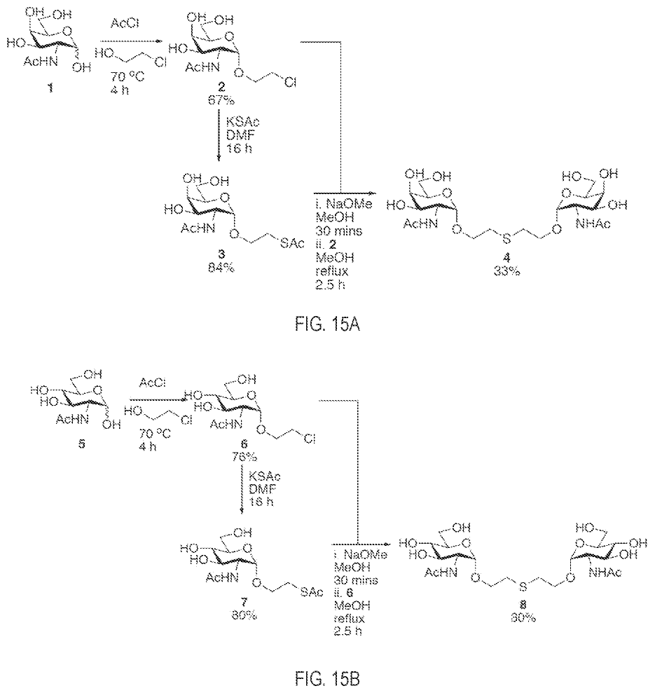

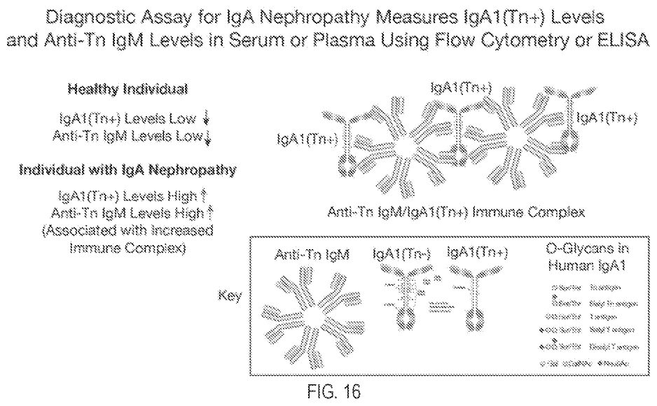

Nevertheless, to further explore the biological activity of the anti-Tn CICs purified from both IgAN patient and healthy controls on mesangial cells, the anti-Tn CICs from both sources were purified. Similar to FIGS. 61 and 6J, cultured cells were prepared with immunodepleted IgAN serum and then the cells were stimulated by adding the same amount of anti-Tn CICs purified from either IgAN sera or from the healthy control sera; for the specificity, HEK293T cells were tested, in parallel with HRMCs, as a control cell line for the cell proliferation assay. It was found that anti-Tn CICs do not stimulate HEK293T cells (FIGS. 14A-14C). Interestingly, the results demonstrate that the anti-Tn CICs from both IgAN and the control serum stimulates the cells as seen by Ki-67 staining (FIGS. 6M-6Q, ID and ID+ anti-Tn CICs). It was then pondered whether α-methylGalNAc, which dissociates the anti-Tn CICs from IgAN patient (FIG. 5F), could inhibit the proliferative activity of the anti-Tn CICs. The result demonstrates that the pretreatment of the anti-Tn CICs purified from IgAN patients with α-methylGalNAc inhibits their proliferative activity toward mesangial cells; importantly, in parallel control experiments, the control compound α-methylGlcNAc did not inhibit the mesangial cell proliferation as expected (FIGS. 60 and 6Q). Interestingly, the use of similarly α-methylGalNAc pretreated anti-Tn CICs purified from healthy control sera did not significantly inhibit mesangial cell proliferation (FIGS. 6P and 6Q). Together, the results demonstrate that the purified anti-Tn CICs, irrespective of IgAN status, can stimulate mesangial cells proliferation and that the glycomimetics specifically inhibit the stimulatory nature of only anti-Tn CICs purified from IgAN patients.

Prior studies suggest that complement plays an important role in IgAN pathogenesis, and IgA1 containing CICs have been observed with complement C3, one of the components of the complement pathway. It has been reported that complement can be activated within the CICs in circulation or after the immune complexes being deposited to glomerular mesangium^{39-41,42} The mass spectrometry analysis of the anti-Tn CICs purified from healthy control sera demonstrated the presence of complement C3 (FIG. 8). To better understand the presence of complement C3 within the purified anti-Tn CICs from IgAN patients, CICs from both healthy and IgAN patient serum were purified and analyzed the materials by SDS-PAGE immunoblots. Purified anti-Tn CICs from IgAN patient sera contain IgM, IgG, Tn(+)IgA1 and significantly higher level of complement C3 compared to CICs purified from healthy controls (FIG. 4R). These results indicate that anti-Tn CICs purified from IgAN patients are different in terms of amounts of complement C3. Also, the significantly higher amount of complement C3 within anti-Tn CICs may explain the differential mesangial cell proliferation by anti-Tn CICs from IgAN compared to healthy control (FIGS. 6I, 6G, 6Q). Together, anti-Tn CICs that were purified from IgAN patient serum contain IgM, Tn(+)IgA1, IgG, and complement C3, suggesting a pathogenic nature of anti-Tn CICs of IgAN patients in their serum.

### Example 6: Diagnostic for IgA Nephropathy

The present disclosure is based, in part, on the discovery of IgM antibodies in human blood that bind to the carbohydrate antigen described herein, termed Tn (GalNAc-α1-Ser/Thr). This antigen is found in subjects with IgAN and is elevated in their blood compared to healthy individuals. These anti-Tn antibodies can bind to the immunoglobulin IgA1, a portion of which has the Tn antigen in the hinge region, the so-called “hinge-region glycans.” The formation of a complex between this IgM and the IgA1 containing the Tn antigen in the hinge-region glycans is presumed to cause IgAN. The buildup of IgA deposits in the kidney inflames and damages the glomeruli, causing the kidneys to leak blood and protein into the urine. The damage may lead to scarring of the nephrons that progresses slowly over many years. Eventually, IgA nephropathy can lead to end-stage kidney disease, sometimes called ESRD, which means the kidneys no longer work well enough to keep a person healthy. When a person's kidneys fail, he or she needs a transplant or blood-filtering treatments called dialysis. The definitive diagnostic is a renal biopsy by a nephrologist and a pathologist finding of deposits of IgA1 in the kidney biopsy. The disease slowly progresses over several years before the pathology becomes evident, usually as blood in the urine.

The diagnostic method described herein required several innovative and novel steps. A form of IgA1 was engineered to express completely the Tn antigen IgA1(Tn+) as a test reagent. This is different from a natural source of to IgA1 expressed by the commercially available Dakiki cells and which lacks the Tn antigen IgA1(Tn−). The IgM discovered in the blood of patients can bind to the IgA1(Tn+) but not natural IgA1(Tn−). A microbead assay has been established to capture anti-Tn IgM antibodies from small amounts of serum and using flow cytometry to measure the amount of IgM that recognizes the Tn antigen. The amount of anti-Tn IgM is significantly elevated in the sera of patients with IgAN compared to sera from healthy individuals. A microbead assay has also been established in which the IgA1(Tn+) or the IgA1(Tn−) proteins are affixed to beads, the anti-Tn IgM in blood that bind to the former but not the latter is measured. Elevations in anti-Tn IgM and/or anti-IgA1(Tn+) are diagnostic for IgAN. This new diagnostic, using either flow cytometry or ELISA, can be useful preclinically, before symptoms of IgAN become apparent. Also, this new diagnostic could be used to monitor treatment of patients, who are often treated by dialysis to remove antibodies, and often given kidney transplants.

### Materials Related to Examples 1-6

### Serum Collection

Blood samples from IgAN patients, biopsy-proven, were obtained either from the Emory Clinic under the approved IRB protocol (IRB00008410) as previously described¹⁷ or from the VUMC Amsterdam, the Netherlands, approved by both the patients and by the Medical-Ethical Committee of the hospital (METC-VUMC) under approved CCMO (NL12328.029.06). Blood samples for healthy donors were obtained from BIDMC hospital under the approved IRB protocol (2016P000008) as well as from the VUMC Amsterdam, under approved CCMO (NL12328.029.06). Additionally, basic donor information is listed in FIG. 1. Collected blood samples were kept at RT (1 h), and centrifuged (2,000 rpm) for 10 min at RT, the resultant supernatants were collected as serum.

### Preparation of Tn(+)Matrix Affinity Resin

A Tn (+) matrix (desialylated bovine submaxillary mucin (Asialo-BSM)) resins were prepared as previously described³⁵. Briefly, ~2 ml of coupled BSM resins (50% slurry) were desialylated using 50 mU neuraminidase (Cat #10269611001, Roche) in 50 mM sodium acetate (pH 5.0) for 1 h at 37° C. (10 rpm). The beads were washed 3× with 10 ml of PBS, and desialylated one more time to completely remove the remaining sialic acid. The prepared desialylated BSM (Asialo-BSM) beads (Tn(+)matrix beads) were kept at 4° C. in PBS for future uses. Beads alone were prepared with no proteins in parallel.

### Affinity Purification of Putative Anti-Tn Antibodies and Western Blot

~50 ml of human sera was mixed with 1 ml of Tn(+)matrix beads (50% slurry in PBS) at 4° C. (10 rpm) overnight. The preparation of Tn(+)matrix beads was washed 6× with 5 ml of chilled 1 M NaCl on a column (Cat #10561284, Pierce™) dropwise (1 ml/min), and the bound material was eluted by 5 ml of chilled 0.1 M glycine-NaOH, pH 10.5 and eluted sample was immediately neutralized with chilled glycine-HCl. 6 ml of eluted fraction was dialyzed in 1 L of PBS using Slide-A-Lyzer™ Dialysis cassettes (10K MWCO, 12 ml, Thermo Fisher Scientific) overnight at 4° C., and concentrated using Amicon #Ultra Centrifugal Filters (Cat #UFC801096, 10K NMWL, Millipore Sigma) down to 0.5 ml. The concentration of eluted protein was determined by BCA assay (Cat #23225, Thermo Fisher Scientific). Each fraction (Input, Flow through (FT), Washes) was proportionally loaded, except elution 10 ml, and analyzed on SDS-PAGE, and stained with Coomassie, or transferred to nitrocellulose membrane (Cat #1704158, Bio-Rad) using Trans-Blot Turbo Transfer System (Bio-Rad). After blocking with 5% (w/vol) non-fat milk (Cat #C701K28, EMD Millipore) in TBST (50 mM Tris-HCl, pH 7.4, 150 mM NaCl, and 0.05% Tween-20) for 1 h at RT, the membranes were incubated with HRP-labeled goat anti-human IgM (m chain) (Cat #5220-0328, KPL), goat anti-human IgG (H+L) (Cat #109-005-003, Jackson ImmunoResearch Laboratories, Inc.), or goat anti-human IgA (α chain) (Cat #5220-0360, KPL) antibodies at 1:10,000 dilution in TBST containing 1% non-fat milk for 1 h at RT. After washing 3× with TBST for 10 min, the signals were analyzed on an Amersham™ Imager 600 (GE Healthcare Life Sciences) using SuperSignal™ West Pico chemiluminescent substrate (Cat #34578, Thermo Scientific). For lectin blot, the membranes were blocked with 5% (w/vol) BSA (Cat #BP1600-1, Fraction V, Fisher BioReagents™) in TBST for 1 h at RT, and incubated with biotinylated VVA (Cat #B-1235-2, Vector Laboratories, diluted to 0.5 mg/ml) in TBST containing 0.5% BSA for 1 h at RT. HRP-labeled streptavidin (Cat #SA-5014, Vector Laboratories) at 1:10,000 dilution in TBST containing 0.5% BSA was used for detection. ~5 ml of Tn(+)matrix beads (50% slurry in PBS) or control beads were incubated with purified anti-Tn antibodies diluted to 1 mg/ml for IgM staining; 4 mg/ml for IgG and IgA staining in PBS for 1 h on ice. The beads were washed 3× with 1 ml of chilled 1 M NaCl each time using centrifugation at 150×g for 30 seconds at 4° C., and incubated with respective Alexa Fluor #488-goat anti-human IgM (Cat #A-21215, Thermo Fisher Scientific), goat anti-human IgG (Cat #A-11013, Thermo Fisher Scientific) or FITC-labeled mouse anti-human IgA1 (Cat #9130-02, Southern Biotech) at 1:400 dilution in PBS for 1 h on ice in the dark. The beads were washed twice with chilled 1 ml of PBS, and analyzed using a microscope (AMG EVOS FL digital inverted microscope, Fisher Scientific, ×100 magnification). Isotype human IgM (Cat #31146, diluted to 1 mg/ml), human IgG (Cat #31154, diluted to 4 mg/ml), or human IgA (Cat #31148, diluted to 4 mg/ml) from Invitrogen in PBS were used as controls. For inhibition assay, purified anti-Tn antibodies were preincubated with 20 mM α-methylGalNAc (Cat #sc-222088, Santa Cruz) in PBS for 30 min on ice. For additional control experiments, other proteins including BSM, BSA, and fetuin were also coupled independently with beads as described in “Preparation of Tn(+)matrix affinity resin”. Beads were incubated with a murine anti-Tn IgM antibody (ReBaGs6 in house³⁵, diluted to 1 mg/ml), or biotinylated VVA (diluted to 1 mg/ml) in PBS, and detected with Alexa Fluor #488-goat anti-mouse IgM (Cat #A-21042, Thermo Fisher Scientific), or streptavidin (Cat #S11223, Invitrogen) at 1:400 dilution in PBS. Isotype mouse IgM (Cat #0101-01, Southern Biotech, diluted to 1 mg/ml) in PBS was used as a control.

### Tn Glycopeptide Microarray

The Tn glycopeptide microarray was prepared as previously described⁷⁷. Briefly, the glycopeptides printed on the microarray is as listed (FIG. 2). Purified anti-Tn antibodies (diluted to 10 mg/ml for IgM, and 40 mg/ml for IgG and IgA detection) in TSM binding buffer (20 mM Tris-HCl, pH 7.4, 150 mM NaCl, 2 mM CaCl₂), 2 mM MgCl₂, with 1% BSA and 0.05% Tween-20) were added to the respective array slides for 1 h at RT. Alexa Fluor #488-labeled goat anti-human IgM or goat anti-human IgG, or FITC-labeled goat anti-human IgA antibody at 1:400 dilution in TSM binding buffer was used for detection of IgM, IgG, and IgA within purified anti-Tn antibodies. Slides were analyzed on a Genepix^{#} 4300A microarray scanner (Molecular Devices). Images were analyzed with quantitation software (GenePix® Pro Microarray Analysis Software Ver. 7, Molecular Devices). For inhibition assay, a purified Tn(+)IgA1 produced from CosmcKO Dakiki cells was utilized (See Generation of CosmcKO Dakiki cells using CRISPR-Cas9 system for generating the antibody) to inhibit anti-Tn antibodies binding to microarray and Tn(−)IgA1 produced from Dakiki cells as a positive control. Purified anti-Tn antibodies (Cl, 10 mg/ml) in TSM binding buffer was preincubated with 1 mg of purified Tn(+)IgA1, or Tn(−)IgA1 for 30 min at RT. After 1 h incubation with the solution on the array, Alexa Fluor #647-labeled goat anti-human IgM antibody (Cat #A-21249, Thermo Fisher Scientific, diluted at 1:400) in TSM binding buffer was used for detection. For inhibition assay with glycomimetics, purified anti-Tn antibodies (C3, 10 mg/ml) in TSM binding buffer was preincubated with 20 mM α-methylGalNAc, or α-methylGlcNAc (Cat #M0257, Sigma), or DiαGalNAc, or DiαGlcNAc synthesized as described in (Synthesis of GalNAc and GlcNAc Dimer (DiαGalNAc and DiαGlcNAc)) for 30 min at RT; after 1 h incubation with the solution on the array, Alexa Fluor #488-labeled goat anti-human IgM (Cat #A-21249, Thermo Fisher Scientific, diluted at 1:400) in TSM binding buffer was used for detection. Heat map and correlation map of binding preferences were analyzed using GLAD (Glycan Array Dashboard).

### CFG Glycan Microarray

The Consortium for Functional Glycomics (CFG) glycan microarray version 5.0 was used (www.functionalglycomics.org)⁷⁸. Briefly, purified anti-Tn antibodies (Cl, diluted to 10 μg/ml) in TSM binding buffer was added to the array slides for 1 h at RT. Alexa Fluor #488-labeled goat anti-human IgM at 1:400 dilution in TSM binding buffer was used for detection. Slides were analyzed as described in “Tn glycopeptide array”

I. Generation of CosmcKO cells: Human B cell line (Dakiki) was purchased from American Type Culture Collection (TIB-206™, ATCC)) and cultured in RPMI 1640 medium (Corning^{#}) supplemented with 20% (vol/vol) fetal bovine serum and 200 units/ml penicillin-streptomycin (Fisher Scientific) at 37° C. and 5% CO₂. The sgRNA sequence (Dharmacon) to target Cosmc gene is described in FIGS. 10A-10E. Dakiki cells were infected by spinoculation. Tn positivity serves as a cell surface marker of CosmcKO. Cells were first selected using drug and then sorted for Tn positive population using ReBaGs6 on a cell sorter (Beckman Coulter, MoFlo Astrios EQs Sorter). The resultant homogenous cell population was termed CosmcKO cells.

Characterization of CosmcKO cells: First, whole-cell extracts from WT were analyzed and CosmcKO Dakiki cells which were resolved on SDS-PAGE and immunoblotted for Cosmc, T-synthase, and “-actin using anti-Cosmc antibody (Cat #sc-271829, H-10, Santa Cruz), anti-T-synthase antibody (Cat #sc-100745, F-31, Santa Cruz), or anti-”-actin antibody (Cat #sc-47778, C4, Santa Cruz) at 1:1000 dilution in TBST with 1% nonfat milk. Secondary detection was performed with HRP-labeled goat anti-mouse IgG (H+L) antibody (Cat #115-035-003, Jackson ImmunoResearch Laboratories, Inc.) at 1:10,000 dilution in TBST containing 0.5% nonfat milk. For lectin blots, cell extracts (~30 μg) were pretreated with 50 mU of neuraminidase in 50 mM Sodium acetate (pH 5.0) for 1 h at 37° C. following the manufacturer's instructions, and analyzed by SDS-PAGE-lectin blots. Biotinylated PNA (Cat #B-1075-5, Vector Laboratories, diluted to 1 μg/ml), or HPA (Cat #L6512, Sigma, diluted to 1 μg/ml) in TBST containing 1% BSA were used as primary reagents. Secondary detection was performed with HRP-labeled streptavidin at 1:10,000 dilution in TBST containing 0.5% BSA.

For the Tn expression levels on the cell surface in CosmcKO Dakiki cell line, cells were incubated with 100 μl of a murine anti-Tn antibody (ReBaGs6, diluted to 1 μg/ml) or mouse isotype IgM (diluted to 1 μg/ml) in PBS for 1 h on ice. Cells were washed twice with 3 ml of cold PBS by centrifugation and incubated with 100 μl of Alexa Fluor #488-labeled goat anti-mouse IgM antibody at 1:400 dilution in PBS for 1 h on ice in the dark. After washing twice with 3 ml of cold PBS by centrifugation, cells were resuspended in 500 μl of PBS, then analyzed on a flow cytometer (FACSCalibur™, Becton Dickinson).

The T-synthase enzyme activity was fluorescently assayed using 4-Methylumbelliferyl 2-acetamido-2-deoxy-α-D-galactopyranoside (Cat #EM04782, Biosynth Carbosynth®, 4MU-α-GalNAc) as an acceptor substrate and UDP-Galactose (MU06699, Biosynth Carbosynth®) as donor sugar as described previously⁷⁹. For a control, α-mannosidase activity was assayed using 4-Methylumbelliferyl α-D-mannopyranoside (Cat #M3657, Sigma-Aldrich, 4MU-α-Man) as an acceptor substrate. The T-synthase activity was normalized as a ratio of T-synthase activity/α-Mannosidase activity in Dakiki WT cells.

The purification procedure of IgA1 from Dakiki cell lines was previously described³.

### Tn(+)IgA1 Binding Study by Purified Anti-Tn Antibodies

CosmcKO Dakiki cells were established as described above. Purification of IgA1 from Dakiki cell line was previously described³⁵. Purified IgA1 with or without extended O-glycans were analyzed by SDS-PAGE-Western blot as described in “Affinity purification of putative anti-Tn antibodies and Western blot” with some modifications. Western blot was performed using the purified anti-Tn antibodies (10 mg/ml) in TBST containing 0.5% BSA as primary antibody. VVA lectin blotting was performed as described above.

### Microbeads Flow Cytometry-Diagnostic Assay

Asialo-BSM (100 mg) and Helix pomatia agglutinin (HPA, Cat #L3382, Sigma, 1 mg) were conjugated to 5×10⁶ Polybead^{#} carboxylate 6.0 micron microspheres (Cat #17141, Polysciences, Inc., 2×10⁷ beads/ml) following the manufacturer's instructions. For detection of anti-Tn IgM in sera ~5×10⁴ Asialo-BSM microbeads were incubated with 100 ml of serum (diluted at 1:100 in PBS) for 1 h on ice. For controls, ~5×10⁴ Asialo-BSM microbeads were incubated with 3.3 mg/ml of isotype human IgM, or serial diluted Asialo-BSM-purified anti-Tn IgM (starting at 3.3 mg/ml) from human sera (Cat #7323901, Lampire Biological Laboratories, Inc. PA) for 1 h on ice. The beads were washed twice with 1 ml of chilled 1M NaCl (beads pelleted 850×g for 30 sec), and incubated with 100 ml of Alexa Fluor #488 labeled goat anti-human IgM (m chain) antibody at 1:400 dilution in PBS for 1 h on ice in the dark. For detection of Tn(+)IgA1 in sera, ~5×10⁴ HPA microbeads were incubated with 100 ml of serum (diluted at 1:100 in PBS) for 1 h on ice. For controls, 5×10⁴ HPA microbeads were incubated with 10 mg/ml of purified Tn(+)IgA1 or Tn(−)IgA1 from Dakiki cell lines for 1 h on ice. The beads were washed twice with 1 ml of cold PBS, and incubated with 100 ml of FITC-labeled mouse anti-human IgA1 antibody at 1:400 dilution in PBS for 1 h on ice in the dark. After washing the beads twice with 1 ml of cold PBS, the beads were resuspended in 500 ml of chilled PBS, and analyzed on a flow cytometer (FACSCalibur™, Becton Dickinson). Total serum IgM in sera was measured by an ELISA kit (Cat #88-50620-22, Thermo Fisher Scientific). Total serum IgA in sera was measured by an ELISA kit (Cat #88-50600-22, Thermo Fisher Scientific).

### Preparation of Blue Native-Agarose Polyacrylamide Gel (BN-APAGE)

Circulating immunocomplexes (CICs) were resolved using BN-APAGE system as previously described³⁴. Samples and unstained native markers (Cat #LC0725, Invitrogen), were mixed with the sample buffer (0.5% Coomassie blue G-250 and 50 mM F-aminocaproic acid in 10 mM Bis-Tris, pH 7.5, at final concentration) just before use, and electrophoresis was performed using 1× anode buffer (50 mM Bis-Tris HCl, pH 7.0) and 1× cathode buffer (50 mM Tricine, 15 mM Bis-Tris, 0.0015% G-250, pH 7.0) for at ~18 h using relatively low voltage and low current (e.g., 15-20 V, ~2 mA, for four gels).

### Western Blot on BN-APAGE

Samples (~0.1 mg) or for plain serum (1:200 dilution in PBS, 10 ml loaded) run on BN-APAGE were transferred onto activated PVDF membrane (Cat #IPVH00010, EMD Millipore, wet-transfer system, 40 V for 2 h). The membranes were quickly destained with 100% methanol, washed with TBST, and blocked with 5% (w/vol) non-fat milk in TBST for 1 h at RT. The blocked membranes were incubated with HRP-labeled goat anti-human IgM (m chain) antibody, or goat anti-mouse IgM (m chain) antibody (Cat #115-035-020, Jackson ImmunoResearch Laboratories, Inc.) at 1:10,000 dilution in TBST containing 1% non-fat milk for 1 h at RT, and the signals were detected onto the autoradiography films (Cat #1141J52, HyBlot CL^{#}, Thomas Scientific) using SuperSignal™ West Pico Chemiluminescent Substrate.

### Immunodepletion Experiment

~10 μl (50% slurry in PBS) of goat anti-human IgA (a chain) agarose beads (Cat #A2691, Sigma), or isotype goat IgG control beads (Cat #ab104155, Abcam) were added to the equal amount of purified anti-Tn CICs (~0.1 μg). After 2 h of incubation in cold room (10 rpm), unbound/depleted fractions were collected after centrifugation at 850×g for 30 sec, and analyzed by Western blot using BN-APAGE system.

### Glycomimetic Treatment of Anti-Tn CICs and BN-APAGE Analysis

For glycomimetic treatment, purified anti-Tn CICs (~0.1 mg) was pretreated with α-methylGalNAc (100 mM), α-methylGlcNAc (100 mM), or mock in PBS for 2 h at 4° C. on a rotator (10 rpm) and the samples were analyzed by BN-APAGE-WB and probed for IgM and IgA.

### ELISA Based Glycomimetic Inhibition Assays

IgA1 glycopeptides (ID18, or ID19 as listed in FIG. 2, 0.01 mg/well) were immobilized using immobilization buffer (NaHCO₃/Na₂CO₃, pH 9.6) in a 96-well plate (Thermo Fisher Scientific, PolySorp) overnight at 4° C. The plate was washed 3× with TSM containing 0.05% tween-20 (TSMT) and added 5% (w/v) BSA in TSMT for 1 h at RT. The plate was incubated with 1 mg/ml of purified anti-Tn CICs from IgAN serum (P3, and pooled with P1-P10; Pmix) or healthy donors (C3, and pooled C1-C10; Cmix) in TSMT containing 0.5% BSA for 1 h at RT. The plate was washed 3× with TSMT and incubated for 1 h with HRP-conjugated goat anti-human IgM at 1:1,000 dilution in TSMT containing 0.5% BSA at RT. The plate was washed 3× with TSMT and developed using TMB substrate solution (Cat #ab171523, Abcam) for 30 min, and the reaction was stopped with 1N HCl. Absorbance (450 nm) was read on an ImageXpress #Pico (Molecular Devices). The half-maximal inhibitory concentration (IC50) was calculated with GraphPad Prism 6.0 (GraphPad Software, Inc.) after subtraction from ID19 as a baseline. For inhibition assay, purified anti-Tn CICs were preincubated with a serial dilution (1/3) of α-methylGalNAc or DiαGalNAc (starting at 10 mM) for 30 min at RT.

### Immunofluorescence on Human Primary Mesangial Cells

Human renal mesangial cells (HRMC) (Cat #4200, ScienCell) were cultured, as described in the protocol of the company, on poly-L-lysine (Cat #0403, 2 mg/cm²)-coated cover strip in 24-well plate. Cells were harvested at ~90% confluency, and fixed with 4% PFA (Cat #50-980-487, Fisher Scientific) with 0.05% triton-X-100 in PBS for 20 min at 4° C. Fixed cells were blocked with 5% (vol/vol) goat serum (Cat #16210064, Thermo Fisher Scientific) in PBS for 1 h at 4° C., and incubated with anti-Tn CICs (lmg/ml), and anti-Vimentin antibody (Cat #sc-6260, V9, mouse IgG, Santa Cruz, diluted to 1:400) in PBS overnight at 4° C. Cells were washed 3× with chilled PBS, and incubated with Alexa Fluor #488-goat anti-human IgM, and Alexa Fluor #633-goat anti-mouse IgG (Cat #A21052, Thermo Fisher Scientific) at 1:400 dilution in PBS for 1 h at 4° C. in the dark. Cells were washed 3× with chilled PBS, and stained with DAPI (Cat #9542, Sigma, diluted to 0.1 mM) in PBS for 10 min at RT, and analyzed by confocal microscope (Zeiss Axioimager Z1, ×630 magnification). Anti-Tn CICs from three healthy controls (C3, C6, and pooled C1-C10; Cmix) and three IgAN (P5, P10, and pooled P1-P10; Pmix) were used on this assay. For isotype control, human IgM, IgG, and IgA (1 mg/ml each), or mouse IgG (Cat #0107-01, Southern Biotech, diluted to 0.5 mg/ml) in PBS were used. All images were taken in three different areas in 24-well plate.

### Cell Surface Staining by Anti-Tn CICs

HRMC cells (~5×10⁵) were stained with 5 mg/ml of anti-Tn CICs purified from IgAN (P5, P10, or Pmix) or healthy control (HC) (C3, C6, or Cmix) for 1 h at 4° C. After washing 3× with 2 ml of cold PBS, cells were incubated with Alexa Fluor #488-goat anti-human IgM (m chain), goat anti-human IgG (H+L), or FITC-labeled mouse anti-human IgA1 at 1:400 dilution in PBS for 1 h on ice. After washing 3× with 2 ml of cold PBS, cells were resuspended in 500 μl of chilled PBS, and analyzed on a flow cytometer (FACSCalibur™, Becton Dickinson). Mean fluorescent intensity, MFI (FL1-H) for each staining was plotted on a graph.

### Cell Proliferation Assay

~500 ml of serum was mixed with 50 ml of Tn(+)matrix beads or mock beads overnight at 4° C. (10 rpm). Supernatant was collected after pelleting the beads at 500×g for 30 sec at 4° C. After two rounds of such sequential anti-Tn CICs immunodepletion (serum-ID) and its control (serum-Mock), collected serum was filtered by syringe filter unit (Cat #SLHP033RS, Millipore Sigma) before use. Cells were cultured at ~90% confluency in 96-well plate, and starved in Mesangial cell media (Cat #4201) with 0.5% FBS (Cat #0010) and 0.05× mesangial cell growth supplement (MsCGS, Cat #4252) from ScienCell for 24 h at 37° C. prior to stimulation. Cells were stimulated with 5% serum-Mock, 5% serum-ID with or without exogenous anti-Tn CICs (50 ng/100 ml/well, total) for 24 h at 37° C. incubator. Cells were fixed with 4% PFA with 0.05% Triton-X-100 in PBS for 20 min at 4° C. Fixed cells were blocked with 5% (vol/vol) goat serum in PBS for 1 h at 4° C., and incubated with anti-Ki-67 antibody (SP6, rabbit IgG, Cat #CRM325B, Biocare Medical, diluted to 1:50) in PBS for 1 h at 4° C. Cells were washed 3× with chilled PBS, and incubated with Alexa Fluor #488-goat anti-rabbit IgG (Cat #A27034, Thermo Fisher Scientific) at 1:400 in PBS for 1 h at 4° C. in the dark. Cells were washed 3× with chilled PBS, and stained with DAPI (diluted to 0.1 mM) in PBS for 10 min at RT, and analyzed using a microscope (AMG EVOS FL digital inverted microscope, Fisher Scientific, ×400 magnification). For inhibition assay, anti-Tn CICs was preincubated with 100 mM α-methylGalNAc, or α-methylGlcNAc in PBS for 30 min at RT. Serum from three healthy controls (C3, C6, and pooled C1-C10; Cmix) and three IgAN (P5, P10, and pooled P1-P10; Pmix) were used in this assay. For cell proliferation studies using plain serum from IgAN patients and healthy control, HRMCs were starved as described above and cells were stimulated with 1, 2.5, or 5% serum with IgAN (Pmix) or healthy control (Cmix) for 24 h in a 37° C. cell culture incubator. For the comparison of cell proliferation studies, cells were stimulated with 2.5% serum with IgAN (P1-P20) or healthy control (C1-C20) for 24 h at 37° C. incubation. One ml of serum with IgAN (P1-P20) or healthy control (C1-C20) was analyzed on SDS-PAGE gel, and stained with Coomassie.

For control cell staining, HEK293T cells (Cat #CRL-3216™, ATCC) were starved in Dulbecco's Modified Eagle's Medium (DMEM) (Cat #10-013-CV, Corning®) with 0.5% FBS for 24 h at 37° C. prior to stimulation. Cells were stimulated with 5% serum with IgAN (mock), CICs-immunodepleted serum (ID), or exogenously adding CICs from IgAN (ID+anti-Tn CICs) for 24 h at 37° C. CO₂ incubator. Serum and anti-Tn CICs (P10, or Pmix) were used in this assay. In parallel, healthy control serum and HRMCs cells were also used as described above. All images were taken in three different areas in 96-well plate, and Ki-67 positive cells were counted. All images were taken in three different areas in 96-well plate, and Ki-67 positive cells were counted.

### Western Blot Analysis for Complement C3 Using Purified Anti-Tn CICs

Purified anti-Tn CICs (~0.1 mg) from three healthy controls (C3, C6, and Cmix (Cm); pooled with C1-C10) and three IgAN patients (P5, P10, and Pmix (Pm); pooled with P1-P10) were analyzed on SDS-PAGE as described. Membranes were blocked with 5% milk in TBST for 1 h at RT, and incubated with an anti-C3 antibody (EPR2988, Cat #ab181147, rabbit IgG, Abcam, diluted at 1:1,000 in TBST) for 1 h at RT. HRP-labeled goat anti-rabbit IgG (Cat #5220-0336, KPL) at 1:10,000 dilution was used for detection. The signals were detected using SuperSignal™ West Pico Chemiluminescent Substrate on an Amersham™ Imager 600.

### Protein Identification by Mass Spectrometry

~5 μg of proteins was resolved using SDS-PAGE, and the appropriate bands were excised and processed for in-gel trypsin treatment as described elsewhere⁸⁰. Next day the supernatant was removed from the gel pieces and 50 μl 50% acetonitrile were added and incubated on the shaker for 10 min at RT. Both supernatants were combined and dried in a speed vac concentrator. The samples were taken up in 15 μl water and diluted 2× in 0.1% formic acid.

2 μl of each sample were used for C18-reversed phase-liquid chromatography-mass spectrometry analysis (C18-RP-LC-MS/MS) using an Ultimate 3000 nano LC coupled to an Orbitrap Fusion Lumos mass spectrometer (both Thermo Fisher). Samples were loaded onto a C18 precolumn (C18 PepMap 100, 300 μm×5 mm, 5 μm, 100 Å, Thermo Fisher Scientific) with 15 μl/min solvent A (0.1% FA in H₂O) for 3 min and separated on a C18 analytical column (picofrit 75 μm ID×150 mm, 3 μm, New Objective) using a linear gradient of 2% to 45% solvent B (80% acetonitrile, 0.1% FA) over 39 min at 400 nL/min. The mass spectrometer was operated under following conditions: The ion source parameters were 2,100 V spray voltage and 200° C. ion transfer tube temperature. MS scans were performed in the orbitrap at a resolution of 60,000 within a scan range of m/z 400-m/z 1,600, a RF lens of 30%, AGC target of 1e5 for a maximum injection time of 50 ms. The top 15 precursors were selected for MS² in a data dependent manner, within a mass range of m/z 400-m/z 1,600 and a minimum intensity threshold of 1e5 and an isolation width of 2 m/z. HCD was performed in stepped collision energy mode of 30% (+/−5%) and detected in the orbitrap with a resolution of 30,000 with the first mass at m/z 120, an AGC target of 2e5 and a maximum injection of 250 ms.

Data analysis was performed against the human Swiss-Prot database (v 2016-05-11) using SEQUEST through proteome discoverer (version 2.1.0.81, Thermo Fisher Scientific) under the following settings: Trypsin (2 missed cleavage sites), precursor mass tolerance of 10 ppm and fragment mass tolerance of 0.02 Da. Dynamic modifications were oxidation on Met, deamidation on Asn, phosphorylation on Ser/Thr and N-terminal acetylation. Static modifications were carbamidomethyl on Cys. False discovery rate was set to 1%. Main protein hits were sorted based on their number of PSMs.

### Synthesis of GalNAc and GlcNAc Dimer (DiαGalNAc and DiαGlcNAc)

The synthesis began with the α-linked glycosidation of chloroethyl linker to N-Acetylgalactosamine (1) by using AcCl at 70° C. Thioacetyl substitution to 2 was successful using KSAc in DMF, which gave 3 in high yield. To dimerize this substrate, the thioacetyl group was removed to expose the thiol using sodium methoxide in methanol and compound 2 was introduced to perform a direct S_{N}2 displacement of the halide to render 4 in 33% yield (DiαGalNAc).

The synthesis of the GlcNAc dimer was repeated in a similar fashion as described above to render 8 from N-Acetylglucosamine (5) with comparable yields.

### General Considerations

All commercially available reagents and solvents were used without further purification. All reactions described were conducted under Argon atmosphere in an oven dried glassware. ¹H NMR, ¹³C NMR, and 2D NMR experiments were performed on a Varian MR 400 MHz spectrometer. The spectral data were reported relative to deuterated peaks (DMSO-d₆, $2.50) and chemical shifts were reported in parts per million (ppm, $). ¹H NMR splitting patterns were designated as singlet (s), doublet (d), triplet (t), doublet of doublets (dd), apparent triplet (app. t.) or multiplet (m) and coupling constants were reported in Hertz (Hz). Thin layer chromatography (TLC) was developed on glass-backed TLC plates (silica gel 60 with a 254 nm fluorescent indicator, 250 mm layer thickness) that were stored over drierite in a desiccator. TLC plates were visualized by coating with ammonium molybdate/cerium (IV) sulfate stain heated mildly on a hot plate. Flash column chromatography was performed on silica gel (32-63 m) with reported solvent systems in v/v ratios. Reverse phase high performance liquid chromatography was performed using Waters (Method A) Gradient Purification System 2767 equipped with Waters 2489 UV/Vis detection module and Waters 2545 Binary Gradient Module using a C18 100 A (250×30 mm, Phenomenex) column (PREPARATORY). Ultraflex II matrix-assisted laser desorption ionization time of flight mass spectrometry (MALDI-TOF MS) was used to analyze samples co-crystallized using super DHB matrix.

2-Chloroethyl 2-acetamido-2-deoxy-α-D-galactopyranoside (2). Compound 2 was made according to a previous protocol⁸¹.

2-Acetylthio 2-acetamido-2-deoxy-α-D-galactopyranoside (3). Potassium thioacetate (3.0 equiv) was slowly added to a solution containing compound 2 (1.0 equiv.) in N, N-dimethylformamide and stirred for 16 h at room temperature. Upon completion, water was added to quench the reaction and concentrated in vacuo. Flash column chromatography using CHCl₃ and MeOH system (v/v 10:1) gave compound 3 in 84% yield as a brown solid (R_{f}=0.1). ¹H NMR (400 MHz, DMSO-d₆): δ 7.50 (d, J=8.4 Hz, 1H), 4.69 (d, J=3.6 Hz, 1H), 4.53-4.56 (m, 2H), 4.43 (br, 1H), 3.99-4.05 (m, 1H), 3.72 (br, 1H), 3.56-3.63 (m, 3H), 3.43-54 (m, 3H), ((td, J=1.6, 6.2 Hz, 2H), 2.33 (s, 3H), 1.84 (s, 3H). ¹³C NMR (125 MHz, DMSO-d₆): δ195.09, 169.65, 97.44, 71.53, 68.04, 67.39, 65.86, 60.60, 49.73, 30.54, 28.38, 22.69.

α-D-2-Ethylthio 2-acetamido-galactopyranosyl α-D-2-Ethylthio 2-acetamido-galactopyranoside (4). Compound 3 dissolved in methanol was added NaOMe (1.2. equiv.) and the reaction was stirred for 30 min at room temperature. Compound 2 (1.1 equiv.) pre-dissolved in MeOH was added to the reaction mixture and refluxed for 2.5 hrs. Reaction was quenched using acetic acid and concentrated in vacuo. Purification using reverse phase high performance liquid chromatography gave compound 4 in 33% yield as a white lyophilizate. (RP-HPLC condition: Solvent A—water+0.1% TFA, Solvent B—acetonitrile. 0-5 min, 2% B, 5-16 min, 10% B, 16-19 min, 15% B, 19-23 min 100% B, 23-25 min, 2% B. 4, Rₜ=8.26 min). ¹H NMR (400 MHz, DMSO-d₆): δ 7.46-7.55 (m, 2H), 4.74 (dd, J=2.8, 16.4 Hz, 2H), 4.43-4.56 (m, 2H), 3.96-4.08 (m, 4H), 3.44-3.77 (m, 10H), 2.67-2.72 (m, 4H), 1.84 (s, 3H). ¹³C NMR (125 MHz, DMSO-d₆): δ 169.62, 97.62, 71.51, 68.12, 67.71, 67.40, 49.67, 43.63, 31.00, 22.68. MALDI-TOF analysis of 4:Calculated [M+H⁺] (C₂₀H₃₆N₂NaO₁₂S⁺)=551.1 m/z. Observed [M+H⁺] (C₂₀H₃₆N₂NaO₁₂S⁺)=551.0 m/z. 2-Chloroethyl 2-acetamido-2-deoxy-α-D-glucopyranoside (6). Compound 6 was made according to a previous protocol⁸¹.

α-D-2-Ethylthio 2-acetamido-glucopyranosyl α-D-2-Ethylthio 2-acetamido-glucopyranoside (8). Potassium thioacetate (3.0 equiv) was slowly added to a solution containing compound 6 (1.0 equiv.) in N,N-dimethylformamide and stirred for 16 h at room temperature. Upon completion, water was added to quench the reaction and concentrated in vacuo. NaOMe (1.2. equiv.) was added to the crude containing compound 7 dissolved in methanol and the reaction was stirred for 30 min at room temperature. Compound 6 (1.1 equiv.) pre-dissolved in MeOH was added to the reaction mixture and refluxed for 2.5 hrs. Reaction was quenched using acetic acid and concentrated in vacuo. Purification using reverse phase high performance liquid chromatography gave compound 8 in 30% yield as a white lyophilizate. (RP-HPLC condition: Solvent A—water+0.1% TFA, Solvent B—acetonitrile. 0-5 min, 2% B, 5-16 min, 10% B, 16-19 min, 15% B, 19-23 min 100% B, 23-25 min, 2% B. 8, Rₜ=8.53 min). ¹H NMR (400 MHz, DMSO-d₆): δ 7.60-7.65 (m, 2H), 4.71-4.77 (m, 2H), 4.61 (d, J=11.2, 1H), 4.47-4.52 (m, 1H), 3.42-3.81 (m, 12H), 3.11-3.21 (m, 2H), 2.67-2.76 (m, 4H), 1.84 (s, 3H). ¹³C NMR (125 MHz, DMSO-d₆): δ 169.58, 96.88, 72.95, 70.79, 70.63, 67.07, 60.85, 53.79, 31.02, 22.62. MALDI-TOF analysis of 8: Calculated [M+H⁺] (C₂₀H₃₆N₂NaO₁₂S⁺)=551.1 m/z. Observed [M+H⁺] (C₂₀H₃₆N₂NaO₁₂S⁺)=551.0 m/z.

### Statistical Analysis

Data were analyzed using Student's 1-test (two-tailed), and the differences were considered statistically significant at p<0.05.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

### ADDITIONAL EMBODIMENTS

1. A method of detecting levels of an antibody in a sample derived from a subject, comprising:
contacting the sample with an antigen, wherein the antibody, if present, binds to the antigen; and
measuring the amount of antibody bound to the antigen.

2. A method of detecting levels of IgM antibody in a sample derived from a subject, comprising:
contacting the sample with an IgA molecule comprising a GalNAc-α1-Ser/Thr antigen (antigen), wherein the IgM antibody, if present, binds to the GalNAc-α1-Ser/Thr antigen; and
measuring the amount of IgM antibody bound to the IgA molecule.

3. The method of embodiment 1, wherein the antibody is an IgM antibody that binds a GalNAc-α1-Ser/Thr antigen.

4. The method of embodiment 1 or embodiment 3, wherein the antigen is an IgA comprising the GalNAc-α1-Ser/Thr antigen.

5. The method of any one of the preceding embodiments, wherein the step of contacting further comprises incubating the sample with the antigen.

6. The method of embodiment 5, wherein the incubating is for a sufficient time for the antigen to bind to the antibody.

7. The method of any one of the preceding embodiments, wherein the antigen is adhered to a solid substrate.

8. The method of embodiment 7, wherein the solid substrate is a microbead.

9. The method of any one of the preceding embodiments, wherein the sample is blood, blood serum, blood plasma, blood fraction, saliva, mucous, urine, or a combination thereof.

10. The method of any one of the preceding embodiments, wherein the sample is blood serum.

11. The method of any one of the preceding embodiments, wherein the step of measuring comprises the use of flow cytometry to detect the amount of antibody bound to the antigen.

12. The method of any one of the preceding embodiments, further comprising separately contacting a second sample with a control antigen and comparing the amount of antibody bound to the antigen to the amount of antibody bound to the control antigen.

13. The method of embodiment 12, wherein the amount of antibody bound to the antigen being higher than the amount of antibody bound to the control antigen is indicative of a positive result.

14. The method of embodiment 13, wherein the positive result is indicative of a diagnosis of IgA nephropathy or Berger's disease.

15. The method of any one of the preceding embodiments, wherein the subject is a mammal.

16. The method of any one of the preceding embodiments, wherein the subject is a human.

17. The method of any one of the preceding embodiments, wherein the subject has, is suspected of having, or is at risk of developing IgA nephropathy or Berger's disease.

18. A method, comprising:
administering to a subject a glycomimetic, wherein the glycomimetic is based on the structure of N-acetylgalactosamine (GalNAc);
wherein:
the subject has, is suspected of having, or is at risk of developing IgA nephropathy or Berger's disease.

19. A method, comprising:
administering to a subject a glycomimetic, wherein the glycomimetic is α-methylGalNAc or DiαGalNAc;
wherein:
the subject has, is suspecting of having, or is at risk of developing IgA nephropathy or Berger's disease.

20. The method of embodiment 18 or embodiment 19, wherein the glycomimetic binds an antibody that is elevated in subjects with IgA nephropathy or Berger's disease.

21. The method of any one of embodiments 18-20, wherein the glycomimetic is a modified αGalNAc, wherein the αGalNAc is modified to change the —OH group(s) and/or N-acetyl group.

22. The method of any one of the preceding embodiments, wherein the glycomimetic is an αGalNAc monosaccharide or disaccharide.

23. The method of embodiment 22, wherein the glycomimetic is an αGalNAc disaccharide.

24. The method of embodiment 23, wherein the αGalNAc disaccharide further comprises linkers linking the two αGalNAc molecules.

25. The method of embodiment 24, wherein the αGalNAc disaccharide comprises a carbohydrate or non-carbohydrate linker.

26. The method of embodiment 24, wherein the linker is a flexible or non-flexible linker.

27. The method of embodiment 24, wherein the linker is a cleavable or stable linker.

28. The method of any one of embodiments 24-27, wherein the glycomimetic comprises one, two, three, four, five, six, seven, eight, nine, or ten linkers.

29. The method of any one of the preceding embodiments, wherein the glycomimetic further comprises one or more constituents on the αGalNAc.

30. The method of embodiment 29, wherein the constituent is a carbohydrate or a non-carbohydrate constituent.

31. The method of any one of the preceding embodiments, wherein the glycomimetic is administered orally, subcutaneously, or intravenously.

32. The method of any one of the preceding embodiments, wherein the glycomimetic is administered as an oral capsule.

33. The method of any one of the preceding embodiments, wherein the glycomimetic, after administration, is absorbed through the gut.

34. The method of any one of the preceding embodiments, wherein the glycomimetic inhibits formation of an immune complex comprising an antibody and an antigen, wherein the antibody is Anti-Tn and the antigen is an IgA comprising a GalNAc-α1-Ser/Thr antigen.

35. The method of embodiment 34, wherein the glycomimetic is capable of dissociating the immune complex.

36. The method of any one of the preceding embodiments, wherein the glycomimetic inhibits proliferation of mesangial cells.

37. The method of any one of the preceding embodiments, wherein the subject is human.

38. The method of any one of the preceding embodiments, wherein administration of the glycomimetic is a treatment for IgA nephropathy or Berger's disease.

39. The method of any one of the preceding embodiments, wherein the glycomimetic reduces symptoms of IgA nephropathy or Berger's disease by 5%, 10%, 20%, 30%, 40%, 50%, 60%, 70%, 80%, 90%, 95%, or 100%.

40. The method of any one of the preceding embodiments, wherein the glycomimetic reduces the risk of developing IgA nephropathy or Berger's disease by 5%, 10%, 20%, 30%, 40%, 50%, 60%, 70%, 80%, 90%, 95%, or 100%.

41. The method of any one of the preceding embodiments, further comprising administering to the subject the glycomimetic and a pharmaceutically acceptable excipient.

### REFERENCES

1. Berger, J. & Hinglais, N. [Intercapillary deposits of IgA-IgG]. J Urol Nephrol (Paris) 74, 694-695 (1968).
2. Wyatt, R. J. & Julian, B. A. IgA nephropathy. N Engl J Med 368, 2402-2414 (2013).
3. Jarrick, S., et al. Mortality in IgA Nephropathy: A Nationwide Population-Based Cohort Study. J Am Soc Nephrol 30, 866-876 (2019).
4. Knoop, T., et al. Mortality in patients with IgA nephropathy. Am J Kidney Dis 62, 883-890 (2013).
5. Geddes, C. C., et al. A tricontinental view of IgA nephropathy. Nephrol Dial Transplant 18, 1541-1548 (2003).
6. Imai, E., et al. Kidney disease screening program in Japan: history, outcome, and perspectives. Clin J Am Soc Nephrol 2, 1360-1366 (2007).
7. Cho, B. S., et al. A nationwide study of mass urine screening tests on Korean school children and implications for chronic kidney disease management. Clin Exp Nephrol 17, 205-210 (2013).
8. Suzuki, H., et al. The pathophysiology of IgA nephropathy. J Am Soc Nephrol 22, 1795-1803 (2011).
9. Knoppova, B., et al. The Origin and Activities of IgA1-Containing Immune Complexes in IgA Nephropathy. Front Immunol 7, 117 (2016).
10. Beerman, I., Novak, J., Wyatt, R. J., Julian, B. A. & Gharavi, A. G. The genetics of IgA nephropathy. Nat Clin Pract Nephrol 3, 325-338 (2007).
11. Torano, A., Tsuzukida, Y., Liu, Y. S. & Putnam, F. W. Location and structural significance of the oligosaccharides in human Ig-A1 and IgA2 immunoglobulins. Proc Natl Acad Sci USA 74, 2301-2305 (1977).
12. Ohyama, Y., Renfrow, M. B., Novak, J. & Takahashi, K. Aberrantly Glycosylated IgA1 in IgA Nephropathy: What We Know and What We Don't Know. J Clin Med 10(2021).
13. Tarelli, E., Smith, A. C., Hendry, B. M., Challacombe, S. J. & Pouria, S. Human serum IgA1 is substituted with up to six O-glycans as shown by matrix assisted laser desorption ionisation time-of-flight mass spectrometry. Carbohydr Res 339, 2329-2335 (2004).
14. Takahashi, K., et al. Clustered O-glycans of IgA1: defining macro- and microheterogeneity by use of electron capture/transfer dissociation. Mol Cell Proteomics 9, 2545-2557 (2010).
15. Mestecky, J., et al. Role of aberrant glycosylation of IgA1 molecules in the pathogenesis of IgA nephropathy. Kidney Blood Press Res 31, 29-37 (2008).
16. Allen, A. C., Harper, S. J. & Feehally, J. Galactosylation of N- and O-linked carbohydrate moieties of IgA1 and IgG in IgA nephropathy. Clin Exp Immunol 100, 470-474 (1995).
17. Lehoux, S., et al. Identification of distinct glycoforms of IgA1 in plasma from patients with immunoglobulin A (IgA) nephropathy and healthy individuals. Mol Cell Proteomics 13, 3097-3113 (2014).
18. Monteiro, R. C., et al. Charge and size of mesangial IgA in IgA nephropathy. Kidney Int 28, 666-671 (1985).
19. Lai, K. N., et al. Activation of podocytes by mesangial-derived TNF-alpha: glomerulo-podocytic communication in IgA nephropathy. Am J Physiol Renal Physiol 294, F945-955 (2008).
20. DeCicco RePass, M. A., et al. Molecular cloning, expression, and characterization of UDP N-acetyl-alpha-d-galactosamine: Polypeptide N-acetylgalactosaminyltransferase 4 from Cryptosporidium parvum. Mol Biochem Parasitol 221, 56-65 (2018).
21. Heimburg-Molinaro, J., et al. Microarray analysis of the human antibody response to synthetic Cryptosporidium glycopeptides. Int J Parasitol 43, 901-907 (2013).
22. Springer, G. F. & Tegtmeyer, H. Origin of anti-Thomsen-Friedenreich (T) and Tn agglutinins in man and in White Leghorn chicks. Br J Haematol 47, 453-460 (1981).
23. Springer, G. F. & Horton, R. E. Blood group isoantibody stimulation in man by feeding blood group-active bacteria. J Clin Invest 48, 1280-1291 (1969).
24. Mestecky, J., Novak, J., Moldoveanu, Z. & Raska, M. IgA nephropathy enigma. Clin Immunol 172, 72-77 (2016).
25. Tomana, M., et al. Galactose-deficient IgA1 in sera of IgA nephropathy patients is present in complexes with IgG. Kidney Int 52, 509-516 (1997).
26. Tomana, M., et al. Circulating immune complexes in IgA nephropathy consist of IgA1 with galactose-deficient hinge region and antiglycan antibodies. J Clin Invest 104, 73-81 (1999).
27. Schena, F. P., et al. Increased serum levels of IgA1-IgG immune complexes and anti-F(ab′)2 antibodies in patients with primary IgA nephropathy. Clin Exp Immunol 77, 15-20 (1989).
28. Yanagihara, T., et al. In vitro-generated immune complexes containing galactose-deficient IgA1 stimulate proliferation of mesangial cells. Results Immunol 2, 166-172 (2012).
29. Tumlin, J. A., Madaio, M. P. & Hennigar, R. Idiopathic IgA nephropathy: pathogenesis, histopathology, and therapeutic options. Clin J Am Soc Nephrol 2, 1054-1061 (2007).
30. Ju, T. & Cummings, R. D. A unique molecular chaperone Cosmc required for activity of the mammalian core 1 beta 3-galactosyltransferase. Proc Natl Acad Sci USA 99, 16613-16618 (2002).
31. Wang, Y., et al. Cosmc is an essential chaperone for correct protein O-glycosylation. Proc Natl Acad Sci USA 107, 9228-9233 (2010).
32. Aryal, R. P., Ju, T. & Cummings, R. D. The endoplasmic reticulum chaperone Cosmc directly promotes in vitro folding of T-synthase. J Biol Chem 285, 2456-2462 (2010).
33. Ju, T., et al. Tn and sialyl-Tn antigens, aberrant O-glycomics as human disease markers. Proteomics Clin Appl 7, 618-631 (2013).
34. Aryal, R. P., et al. Macromolecular Assemblies of the Mammalian Circadian Clock. Mol Cell 67, 770-782 e776 (2017).
35. Matsumoto, Y., et al. Identification of Tn antigen 0-GalNAc-expressing glycoproteins in human carcinomas using novel anti-Tn recombinant antibodies. Glycobiology 30, 282-300 (2020).
36. Novak, J., et al. IgA1-containing immune complexes in IgA nephropathy differentially affect proliferation of mesangial cells. Kidney Int 67, 504-513 (2005).
37. Moura, I. C., et al. Glycosylation and size of IgA1 are essential for interaction with mesangial transferrin receptor in IgA nephropathy. J Am Soc Nephrol 15, 622-634 (2004).
38. Tam, K. Y., et al. Macromolecular IgA1 taken from patients with familial IgA nephropathy or their asymptomatic relatives have higher reactivity to mesangial cells in vitro. Kidney Int 75, 1330-1339 (2009).
39. Czerkinsky, C., et al. Circulating immune complexes and immunoglobulin A rheumatoid factor in patients with mesangial immunoglobulin A nephropathies. J Clin Invest 77, 1931-1938 (1986).
40. Rizk, D. V., et al. The Emerging Role of Complement Proteins as a Target for Therapy of IgA Nephropathy. Front Immunol 10, 504 (2019).
41. Maillard, N., et al. Current Understanding of the Role of Complement in IgA Nephropathy. J Am Soc Nephrol 26, 1503-1512 (2015).
42. Jennette, J. C. The immunohistology of IgA nephropathy. Am J Kidney Dis 12, 348-352 (1988).
43. Woodroffe, A. J., et al. Immunologic studies in IgA nephropathy. Kidney Int 18, 366-374 (1980).
44. Eison, T. M., et al. Association of IgG co-deposition with serum levels of galactose-deficient IgA1 in pediatric IgA nephropathy. Clin Nephrol 78, 465-469 (2012).
45. Katafuchi, R., Nagae, H., Masutani, K., Tsuruya, K. & Mitsuiki, K. Comprehensive evaluation of the significance of immunofluorescent findings on clinicopathological features in IgA nephropathy. Clin Exp Nephrol 23, 169-181 (2019).
46. Heybeli, C., et al. Clinical significance of mesangial IgM deposition in patients with IgA nephropathy. Clin Exp Nephrol 23, 371-379 (2019).
47. Bellur, S. S., et al. Immunostaining findings in IgA nephropathy: correlation with histology and clinical outcome in the Oxford classification patient cohort. Nephrol Dial Transplant 26, 2533-2536 (2011).
48. Roberts, I. S. Pathology of IgA nephropathy. Nat Rev Nephrol 10, 445-454 (2014).
49. Wada, Y., et al. Clinical significance of IgG deposition in the glomerular mesangial area in patients with IgA nephropathy. Clin Exp Nephrol 17, 73-82 (2013).
50. Zeis, P. M., et al. Glomerulopathy with mesangial IgM deposits: long-term follow up of 64 children. Pediatr Int 43, 287-292 (2001).
51. Moldoveanu, Z., et al. Experimental evidence of pathogenic role of IgG autoantibodies in IgA nephropathy. J Autoimmun 118, 102593 (2021).
52. Rizk, D. V., et al. Glomerular Immunodeposits of Patients with IgA Nephropathy Are Enriched for IgG Autoantibodies Specific for Galactose-Deficient IgA1. J Am Soc Nephrol 30, 2017-2026 (2019).
53. Cejas, R. B., Lorenz, V., Garay, Y. C. & Irazoqui, F. J. Biosynthesis of O—N-acetylgalactosamine glycans in the human cell nucleus. J Biol Chem 294, 2997-3011 (2019).
54. Giannakakis, K., Feriozzi, S., Perez, M., Faraggiana, T. & Muda, A. O. Aberrantly glycosylated IgA1 in glomerular immune deposits of IgA nephropathy. J Am Soc Nephrol 18, 3139-3146 (2007).
55. Neugut, Y. D. & Kiryluk, K. Genetic Determinants of IgA Nephropathy: Western Perspective. Semin Nephrol 38, 443-454 (2018).
56. Ju, T., Brewer, K., D'Souza, A., Cummings, R. D. & Canfield, W. M. Cloning and expression of human core 1 beta1,3-galactosyltransferase. J Biol Chem 277, 178-186 (2002).
57. Kiryluk, K., et al. GWAS for serum galactose-deficient IgA1 implicates critical genes of the O-glycosylation pathway. PLoS Genet 13, e1006609 (2017).
58. Hu, S., et al. Increased miR-374b promotes cell proliferation and the production of aberrant glycosylated IgA1 in B cells of IgA nephropathy. FEBS Lett 589, 4019-4025 (2015).
59. Yamada, K., et al. Down-regulation of core 1 beta1,3-galactosyltransferase and Cosmc by Th2 cytokine alters O-glycosylation of IgA1. Nephrol Dial Transplant 25, 3890-3897 (2010).
60. Qin, W., et al. Peripheral B lymphocyte beta1,3-galactosyltransferase and chaperone expression in immunoglobulin A nephropathy. J Intern Med 258, 467-477 (2005).
61. Sun, Q., Zhang, J., Zhou, N., Liu, X. & Shen, Y. DNA methylation in Cosmc promoter region and aberrantly glycosylated IgA1 associated with pediatric IgA nephropathy. PLoS One 10, e0112305 (2015).
62. Gale, D. P., et al. Galactosylation of IgA1 Is Associated with Common Variation in C1GALT1. J Am Soc Nephrol 28, 2158-2166 (2017).
63. Malycha, F., et al. No evidence for a role of cosmc-chaperone mutations in European IgA nephropathy patients. Nephrol Dial Transplant 24, 321-324 (2009).
64. Buck, K. S., et al. B-cell O-galactosyltransferase activity, and expression of O-glycosylation genes in bone marrow in IgA nephropathy. Kidney Int 73, 1128-1136 (2008).
65. Coppo, R., et al. Circulating immune complexes containing IgA, IgG and IgM in patients with primary IgA nephropathy and with Henoch-Schoenlein nephritis. Correlation with clinical and histologic signs of activity. Clin Nephrol 18, 230-239 (1982).
66. Suzuki, K., et al. Incidence of latent mesangial IgA deposition in renal allograft donors in Japan. Kidney Int 63, 2286-2294 (2003).
67. Sinniah, R. Occurrence of mesangial IgA and IgM deposits in a control necropsy population. J Clin Pathol 36, 276-279 (1983).
68. Ernst, B. & Magnani, J. L. From carbohydrate leads to glycomimetic drugs. Nat Rev Drug Discov 8, 661-677 (2009).
69. Mannik, M. Mechanisms of tissue deposition of immune complexes. J Rheumatol Suppl 14 Supp113, 35-42 (1987).
70. Mannik, M. Pathophysiology of circulating immune complexes. Arthritis Rheum 25, 783-787 (1982).
71. Brasil, S., et al. CDG Therapies: From Bench to Bedside. Int J Mol Sci 19(2018).
72. Park, J. H., et al. L-Fucose treatment of FUT8-CDG. Mol Genet Metab Rep 25, 100680 (2020).
73. Marquardt, T., et al. Correction of leukocyte adhesion deficiency type II with oral fucose. Blood 94, 3976-3985 (1999).
74. Cagdas, D., et al. A novel mutation in leukocyte adhesion deficiency type II/CDGIIc. J Clin Immunol 34, 1009-1014 (2014).
75. Harms, H. K., et al. Oral mannose therapy persistently corrects the severe clinical symptoms and biochemical abnormalities of phosphomannose isomerase deficiency. Acta Paediatr 91, 1065-1072 (2002).
76. Hendriksz, C. J., et al. Successful treatment of carbohydrate deficient glycoprotein syndrome type 1b with oral mannose. Arch Dis Child 85, 339-340 (2001).
77. Borgert, A., et al. Deciphering structural elements of mucin glycoprotein recognition. ACS Chem Biol 7, 1031-1039 (2012).
78. Heimburg-Molinaro, J., Song, X., Smith, D. F. & Cummings, R. D. Preparation and analysis of glycan microarrays. Curr Protoc Protein Sci Chapter 12, Unit12 10 (2011).
79. Ju, T. & Cummings, R. D. A fluorescence-based assay for Core 1 beta3galactosyltransferase (T-synthase) activity. Methods Mol Biol 1022, 15-28 (2013).
80. Plomp, R., et al. Site-specific N-glycosylation analysis of human immunoglobulin e. J Proteome Res 13, 536-546 (2014).
81. Wang, Q., Ekanayaka, S. A., Wu, J., Zhang, J. & Guo, Z. Synthetic and immunological studies of 5′-N-phenylacetyl sTn to develop carbohydrate-based cancer vaccines and to explore the impacts of linkage between carbohydrate antigens and carrier proteins. Bioconjug Chem 19, 2060-2067 (2008).

What is claimed is:

1. A method of detecting levels of an antibody in a sample derived from a subject, comprising:
contacting the sample with an antigen, wherein the antibody, if present, binds to the antigen; and
measuring the amount of antibody bound to the antigen.

2. A method of detecting levels of IgM antibody in a sample derived from a subject, comprising:
contacting the sample with an IgA molecule comprising a GalNAc-α1-Ser/Thr antigen (antigen), wherein the IgM antibody, if present, binds to the GalNAc-α1-Ser/Thr antigen; and
measuring the amount of IgM antibody bound to the IgA molecule.

3. The method of claim 1, wherein the antibody is an IgM antibody that binds a GalNAc-α1-Ser/Thr antigen.

4. The method of claim 1 or claim 3, wherein the antigen is an IgA comprising the GalNAc-α1-Ser/Thr antigen.

5. The method of claim 1 or 2, wherein the step of contacting further comprises incubating the sample with the antigen.

6. The method of claim 5, wherein the incubating is for a sufficient time for the antigen to bind to the antibody.

7. The method of claim 1, wherein the antigen is adhered to a solid substrate.

8. The method of claim 7, wherein the solid substrate is a microbead.

9. The method of claim 1 or 2, wherein the sample is blood, blood serum, blood plasma, blood fraction, saliva, mucous, urine, or a combination thereof.

10. The method of claim 1 or 2, wherein the sample is blood serum.

11. The method of claim 1 or 2, wherein the step of measuring comprises the use of flow cytometry to detect the amount of antibody bound to the antigen.

12. The method of claim 1 or 2, further comprising separately contacting a second sample with a control antigen and comparing the amount of antibody bound to the antigen to the amount of antibody bound to the control antigen.

13. The method of claim 12, wherein the amount of antibody bound to the antigen being higher than the amount of antibody bound to the control antigen is indicative of a positive result.

14. The method of claim 13, wherein the positive result is indicative of a diagnosis of IgA nephropathy or Berger's disease.

15. The method of claim 1 or 2, wherein the subject is a mammal.

16. The method of claim 15, wherein the subject is a human.

17. The method of claim 16, wherein the subject has, is suspected of having, or is at risk of developing IgA nephropathy or Berger's disease.

18. A method, comprising:
administering to a subject a glycomimetic, wherein the glycomimetic is based on the structure of N-acetylgalactosamine (GalNAc);
wherein:
the subject has, is suspected of having, or is at risk of developing IgA nephropathy or Berger's disease.

19. A method, comprising:
administering to a subject a glycomimetic, wherein the glycomimetic is α-methylGalNAc or DiαGalNAc;
wherein:
the subject has, is suspecting of having, or is at risk of developing IgA nephropathy or Berger's disease.

20. The method of claim 18 or claim 19, wherein the glycomimetic binds an antibody that is elevated in subjects with IgA nephropathy or Berger's disease.

21. The method of claim 20, wherein the glycomimetic is a modified αGalNAc, wherein the αGalNAc is modified to change the —OH group(s) and/or N-acetyl group.

22. The method of claim 20, wherein the glycomimetic is an αGalNAc monosaccharide or disaccharide.

23. The method of claim 22, wherein the glycomimetic is an αGalNAc disaccharide.

24. The method of claim 23, wherein the αGalNAc disaccharide further comprises linkers linking the two αGalNAc molecules.

25. The method of claim 24, wherein the αGalNAc disaccharide comprises a carbohydrate or non-carbohydrate linker.

26. The method of claim 24, wherein the linker is a flexible or non-flexible linker.

27. The method of claim 24, wherein the linker is a cleavable or stable linker.

28. The method of claim 24, wherein the glycomimetic comprises one, two, three, four, five, six, seven, eight, nine, or ten linkers.

29. The method of claim 21, wherein the glycomimetic further comprises one or more constituents on the αGalNAc.

30. The method of claim 29, wherein the constituent is a carbohydrate or a non-carbohydrate constituent.

31. The method of claim 18 or 19, wherein the glycomimetic is administered orally, subcutaneously, or intravenously.

32. The method of claim 31, wherein the glycomimetic is administered as an oral capsule.

33. The method of claim 31, wherein the glycomimetic, after administration, is absorbed through the gut.

34. The method of claim 18 or 19, wherein the glycomimetic inhibits formation of an immune complex comprising an antibody and an antigen, wherein the antibody is Anti-Tn and the antigen is an IgA comprising a GalNAc-α1-Ser/Thr antigen.

35. The method of claim 34, wherein the glycomimetic is capable of dissociating the immune complex.

36. The method of claim 18 or 19, wherein the glycomimetic inhibits proliferation of mesangial cells.

37. The method of claim 18 or 19, wherein the subject is human.

38. The method of claim 18 or 19, wherein administration of the glycomimetic is a treatment for IgA nephropathy or Berger's disease.

39. The method of claim 18 or 19, wherein the glycomimetic reduces symptoms of IgA nephropathy or Berger's disease by 5%, 10%, 20%, 30%, 40%, 50%, 60%, 70%, 80%, 90%, 95%, or 100%.

40. The method of claim 18 or 19, wherein the glycomimetic reduces the risk of developing IgA nephropathy or Berger's disease by 5%, 10%, 20%, 30%, 40%, 50%, 60%, 70%, 80%, 90%, 95%, or 100%.

41. The method of claim 18 or 19, further comprising administering to the subject the glycomimetic and a pharmaceutically acceptable excipient.


SEQUENCE LISTING (48 sequences)

SEQ ID NO: 1 - protein, 7 aa, synthetic construct
  MOD_RES 2: note = N-acetylgalactosamine-alpha1-Serine/Threonine antigen residue
PTTTPLK

SEQ ID NO: 2 - protein, 7 aa, synthetic construct
  MOD_RES 3: note = N-acetylgalactosamine-alpha1-Serine/Threonine antigen residue
PTTTPLK

SEQ ID NO: 3 - protein, 7 aa, synthetic construct
  MOD_RES 4: note = N-acetylgalactosamine-alpha1-Serine/Threonine antigen residue
PTTTPLK

SEQ ID NO: 4 - protein, 7 aa, synthetic construct
  MOD_RES 2: note = N-acetylgalactosamine-alpha1-Serine/Threonine antigen residue
  MOD_RES 3: note = N-acetylgalactosamine-alpha1-Serine/Threonine antigen residue
PTTTPLK

SEQ ID NO: 5 - protein, 7 aa, synthetic construct
  MOD_RES 2: note = N-acetylgalactosamine-alpha1-Serine/Threonine antigen residue
  MOD_RES 4: note = N-acetylgalactosamine-alpha1-Serine/Threonine antigen residue
PTTTPLK

SEQ ID NO: 6 - protein, 7 aa, synthetic construct
  MOD_RES 3: note = N-acetylgalactosamine-alpha1-Serine/Threonine antigen residue
  MOD_RES 4: note = N-acetylgalactosamine-alpha1-Serine/Threonine antigen residue
PTTTPLK

SEQ ID NO: 7 - protein, 7 aa, synthetic construct
  MOD_RES 2: note = N-acetylgalactosamine-alpha1-Serine/Threonine antigen residue
  MOD_RES 3: note = N-acetylgalactosamine-alpha1-Serine/Threonine antigen residue
  MOD_RES 4: note = N-acetylgalactosamine-alpha1-Serine/Threonine antigen residue
PTTTPLK

SEQ ID NO: 8 - protein, 7 aa, synthetic construct
PTTTPLK

SEQ ID NO: 9 - protein, 9 aa, synthetic construct
PPTTTTKKP

SEQ ID NO: 10 - protein, 16 aa, synthetic construct
  MOD_RES 9: note = N-acetylgalactosamine-alpha1-Serine/Threonine antigen residue
GTTPSPVPTTSTTSAP

SEQ ID NO: 11 - protein, 13 aa, synthetic construct
  MOD_RES 7: note = N-acetylgalactosamine-alpha1-Serine/Threonine antigen residue
PTTDSTTPAPTTK

SEQ ID NO: 12 - protein, 13 aa, synthetic construct
PTTDSTTPAPTTK

SEQ ID NO: 13 - protein, 9 aa, synthetic construct
  MOD_RES 3: note = N-acetylgalactosamine-alpha1-Serine/Threonine antigen residue
  MOD_RES 4: note = N-acetylgalactosamine-alpha1-Serine/Threonine antigen residue
  MOD_RES 5: note = N-acetylgalactosamine-alpha1-Serine/Threonine antigen residue
  MOD_RES 6: note = N-acetylgalactosamine-alpha1-Serine/Threonine antigen residue
PPTTTTKKP

SEQ ID NO: 14 - protein, 10 aa, synthetic construct
  MOD_RES 6: note = N-acetylgalactosamine-alpha1-Serine/Threonine antigen residue
TSAPDTRDAP

SEQ ID NO: 15 - protein, 10 aa, synthetic construct
TSAPDTRDAP

SEQ ID NO: 16 - protein, 8 aa, synthetic construct
APGSTAPP

SEQ ID NO: 17 - protein, 8 aa, synthetic construct
APGSTAPP

SEQ ID NO: 18 - protein, 12 aa, synthetic construct
  MOD_RES 6: note = N-acetylgalactosamine-alpha1-Serine/Threonine antigen residue
  MOD_RES 9: note = N-acetylgalactosamine-alpha1-Serine/Threonine antigen residue
  MOD_RES 11: note = N-acetylgalactosamine-alpha1-Serine/Threonine antigen residue
KPVPSTPPTPSC

SEQ ID NO: 19 - protein, 12 aa, synthetic construct
KPVPSTPPTPSC

SEQ ID NO: 20 - protein, 12 aa, synthetic construct
  MOD_RES 5: note = N-acetylgalactosamine-alpha1-Serine/Threonine antigen residue
KPVPSTPPTPSC

SEQ ID NO: 21 - protein, 12 aa, synthetic construct
  MOD_RES 4: note = N-acetylgalactosamine-alpha1-Serine/Threonine antigen residue
  MOD_RES 7: note = N-acetylgalactosamine-alpha1-Serine/Threonine antigen residue
  MOD_RES 9: note = N-acetylgalactosamine-alpha1-Serine/Threonine antigen residue
  MOD_RES 11: note = N-acetylgalactosamine-alpha1-Serine/Threonine antigen residue
KPSTPPTPSPSC

SEQ ID NO: 22 - protein, 12 aa, synthetic construct
KPSTPPTPSPSC

SEQ ID NO: 23 - protein, 12 aa, synthetic construct
  MOD_RES 2: note = N-acetylgalactosamine-alpha1-Serine/Threonine antigen residue
  MOD_RES 5: note = N-acetylgalactosamine-alpha1-Serine/Threonine antigen residue
  MOD_RES 7: note = N-acetylgalactosamine-alpha1-Serine/Threonine antigen residue
  MOD_RES 9: note = N-acetylgalactosamine-alpha1-Serine/Threonine antigen residue
KTPPTPSPSTPC

SEQ ID NO: 24 - protein, 12 aa, synthetic construct
KTPPTPSPSTPC

SEQ ID NO: 25 - protein, 12 aa, synthetic construct
  MOD_RES 10: note = N-acetylgalactosamine-alpha1-Serine/Threonine antigen residue
KTPPTPSPSTPC

SEQ ID NO: 26 - protein, 12 aa, synthetic construct
  MOD_RES 3: note = N-acetylgalactosamine-alpha1-Serine/Threonine antigen residue
  MOD_RES 5: note = N-acetylgalactosamine-alpha1-Serine/Threonine antigen residue
  MOD_RES 7: note = N-acetylgalactosamine-alpha1-Serine/Threonine antigen residue
  MOD_RES 11: note = N-acetylgalactosamine-alpha1-Serine/Threonine antigen residue
KPTPSPSTPPTC

SEQ ID NO: 27 - protein, 12 aa, synthetic construct
KPSPSTPPTPSC

SEQ ID NO: 28 - protein, 12 aa, synthetic construct
  MOD_RES 3: note = N-acetylgalactosamine-alpha1-Serine/Threonine antigen residue
  MOD_RES 5: note = N-acetylgalactosamine-alpha1-Serine/Threonine antigen residue
  MOD_RES 9: note = N-acetylgalactosamine-alpha1-Serine/Threonine antigen residue
KPSPSTPPTPSC

SEQ ID NO: 29 - protein, 12 aa, synthetic construct
KPSTPPTPSPSC

SEQ ID NO: 30 - protein, 12 aa, synthetic construct
  MOD_RES 3: note = N-acetylgalactosamine-alpha1-Serine/Threonine antigen residue
  MOD_RES 7: note = N-acetylgalactosamine-alpha1-Serine/Threonine antigen residue
KPSTPPTPSPSC

SEQ ID NO: 31 - protein, 12 aa, synthetic construct
KPSTPPTPSPSC

SEQ ID NO: 32 - protein, 12 aa, synthetic construct
  MOD_RES 4: note = N-acetylgalactosamine-alpha1-Serine/Threonine antigen residue
  MOD_RES 9: note = N-acetylgalactosamine-alpha1-Serine/Threonine antigen residue
KPSTPPTPSPSC

SEQ ID NO: 33 - protein, 12 aa, synthetic construct
  MOD_RES 9: note = N-acetylgalactosamine-alpha1-Serine/Threonine antigen residue
KPSTPPTPSPSC

SEQ ID NO: 34 - protein, 12 aa, synthetic construct
  MOD_RES 11: note = N-acetylgalactosamine-alpha1-Serine/Threonine antigen residue
KPSTPPTPSPSC

SEQ ID NO: 35 - protein, 12 aa, synthetic construct
  MOD_RES 4: note = N-acetylgalactosamine-alpha1-Serine/Threonine antigen residue
KPSTPPTPSPSC

SEQ ID NO: 36 - DNA, 56 bp, Homo sapiens
  source 1..56: mol_type = genomic DNA
tatcttccaaatcatccctccctgttcaggacacttttctgggatattcacaaggc

SEQ ID NO: 37 - DNA, 42 bp, Homo sapiens
  source 1..42: mol_type = genomic DNA
tatcttccaaatcatccctcttttctgggatattgagaaggc

SEQ ID NO: 38 - DNA, 55 bp, Homo sapiens
  source 1..55: mol_type = genomic DNA
tatcttccaaatcatccctccctttcaggacacttttctgggatattgagaaggc

SEQ ID NO: 39 - DNA, 58 bp, Homo sapiens
  source 1..58: mol_type = genomic DNA
tatcttccaaatcatccctccctttgttcaggacacttttctgggatattgagaaggc

SEQ ID NO: 40 - DNA, 40 bp, Homo sapiens
  source 1..40: mol_type = genomic DNA
tatcttccaaatcatcccttttctgggatattgagaaggc

SEQ ID NO: 41 - DNA, 58 bp, Homo sapiens; identical to SEQ ID NO: 39
  source 1..58: mol_type = genomic DNA

SEQ ID NO: 42 - DNA, 57 bp, Homo sapiens
  source 1..57: mol_type = genomic DNA
tatcttccaaatcatccctcccttgttcaggacacttttctgggatattgagaaggc

SEQ ID NO: 43 - DNA, 57 bp, Homo sapiens; identical to SEQ ID NO: 42
  source 1..57: mol_type = genomic DNA

SEQ ID NO: 44 - DNA, 11 bp, Homo sapiens
  source 1..11: mol_type = genomic DNA
tatcttaaggc

SEQ ID NO: 45 - DNA, 58 bp, Homo sapiens; identical to SEQ ID NO: 39
  source 1..58: mol_type = genomic DNA

SEQ ID NO: 46 - DNA, 36 bp, Homo sapiens
  source 1..36: mol_type = genomic DNA
tatcttcaggacacttttctgggatattgagaaggc

SEQ ID NO: 47 - DNA, 57 bp, Homo sapiens; identical to SEQ ID NO: 42
  source 1..57: mol_type = genomic DNA

SEQ ID NO: 48 - DNA, 57 bp, Homo sapiens; identical to SEQ ID NO: 42
  source 1..57: mol_type = genomic DNA